(19) United States
(12) Patent Application Publication
(10) Pub. No.: US 2024/0207073 A1
(43) Pub. Date: Jun. 27, 2024

(54) GAIT CONTROLS FOR POWERED KNEE PROSTHESIS

(71) Applicant: Össur Iceland ehf, Reykjavik (IS)

(72) Inventors: Arni Einarsson, Reykjavik (IS); David Jonsson, Reykjavik (IS); Selina Zwerver, Reykjavik (IS)

(21) Appl. No.: 18/392,486

(22) Filed: Dec. 21, 2023

Related U.S. Application Data
(60) Provisional application No. 63/476,450, filed on Dec. 21, 2022.

(51) Int. Cl. A61F 2/76; A61F 2/64; A61F 2/70

(52) U.S. Cl. CPC A61F 2/76; A61F 2/64; A61F 2/70; A61F 2002/7625; A61F 2002/7645

(57) ABSTRACT

A powered prosthetic or orthotic device (POD) having gait controls is disclosed. The POD may include a control system that modifies the POD's gait controls based on the activity of the user. For example, the POD may have specific gait controls for a slow walk activity, and different gait controls for a fast walk activity. Each activity may have one or more phases and one or more subphases. For example, a standing-up activity may include a stance phase and may include an extension and braking subphase. The POD may modify the gait controls based on the phase and sub-phase of the activity. Thus, the behavior of the POD may be improved for the current activity of the user and may provide mobility, safety, and support in a variety of different terrains and tasks.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims the priority benefit of U.S. Provisional Application No. 63/476,450 filed Dec. 21, 2022, the entirety of which is incorporated herein by reference.

### BACKGROUND

### Field

The present application relates to features associated with a control system for managing operation of a lower-limb prosthesis when in use by an amputee.

### Description of the Related Art

Lower-limb prosthetic devices have been around for a long time, but the technology in the field has progressed slowly. Thus, there is a large gap between the ambulatory efficiency and performance observed on an amputee wearing a state-of-the-art device and what is observed on non-amputees. In recent years, progress has been made towards restoring the lost lower-limb function and performance through the integration of powered actuators in lower-limb prosthetic devices. In contrast to traditional hinges or passive mechanisms used in lower-limb prosthetic devices, powered actuators have the capacity to generate net positive mechanical power. The generation of power allows the lower-limb prosthetic devices to more closely match the human lower-limb joint characteristics.

While the addition of power generation capabilities to lower-limb prosthetics allow prosthetics to bridge a functional gap observed on current commercial devices, they also create a need for more advanced control systems to properly manage the power generation and synergy between the prosthetic device and remaining anatomical structures. Control systems for lower-limb powered prosthetic devices may be designed to manage the delivery of the positive mechanical power, while not creating artificial constraints or limitations in the device usability.

### SUMMARY

The present disclosure describes example systems, methods, and apparatuses related to a prosthetic or orthotic device including: a prosthetic or orthotic device including: a first limb member; a second limb member coupled to the first limb member at a joint; an actuator coupled to the first limb member and the second limb member and configured to actuate the first limb member relative to the second limb member; and a controller configured to control the actuator, the controller further configured to: based at least in part on a determination that an angle between the first limb member and the second limb member satisfies an angle parameter threshold, select a standing-up mode for a controller mode of the controller, wherein in the standing-up mode, the controller causes the actuator to exhibit an extension behavior during a first portion of a stance phase and exhibit a braking behavior during a second portion of the stance phase.

In some aspects, the techniques described herein relate to a prosthetic or orthotic device, wherein the prosthetic or orthotic device further includes of a ground contact sensor and a torque sensor.

In some aspects, the techniques described herein relate to a prosthetic or orthotic device, wherein the controller is further configured to: based at least in part on a determination that the prosthetic or orthotic device is under a load that satisfies a load threshold, select the standing-up mode for the controller mode of the controller.

In some aspects, the techniques described herein relate to a prosthetic or orthotic device, wherein the prosthetic or orthotic device further includes of one or more position sensors.

In some aspects, the techniques described herein relate to a prosthetic or orthotic device, wherein the angle parameter threshold is a first parameter threshold, and wherein the second portion of the stance phase begins when the controller determines that the angle between the first limb member and the second limb member satisfies a second angle parameter threshold.

In some aspects, the techniques described herein relate to a prosthetic or orthotic device, wherein the controller is further configured to: based at least in part on a determination that a torque value does not satisfies a torque threshold, cause the extension behavior to correspond to a force following behavior.

In some aspects, the techniques described herein relate to a prosthetic or orthotic device, wherein the controller is further configured to: based at least in part on a determination that an absolute orientation of the first limb member satisfies an orientation threshold, select the standing-up mode for the controller mode of the controller.

In some aspects, the techniques described herein relate to a prosthetic or orthotic device, wherein the controller is further configured to: based at least in part on a determination that a torque value satisfies a torque threshold, cause the extension behavior to correspond to a power injection behavior.

In some aspects, the techniques described herein relate to a prosthetic or orthotic device including: a first limb member; a second limb member coupled to the first limb member at a joint; an actuator coupled to the first limb member and the second limb member and configured to actuate the first limb member relative to the second limb member; and a controller configured to control the actuator via a control map, wherein the control map includes of gait activities and gait activity transitions, wherein each of the gait activities and the gait activity transitions correspond to one or more actuator behaviors, and wherein the controller is further configured to: based at least in part on a determine that one or more entry conditions of a gait activity are satisfied, select a gait activity transition to transition from a first gait activity to a second gait activity, wherein the second gait activity is determined based on the one or more entry conditions that were satisfied.

In some aspects, the techniques described herein relate to a prosthetic or orthotic device, wherein the prosthetic or orthotic device further includes of a ground contact sensor and a torque sensor.

In some aspects, the techniques described herein relate to a prosthetic or orthotic device, wherein one of the gait activity transitions is a sit-to-stand transition, wherein the controller uses the sit-to-stand transition to transition from a sit gait activity to a stand gait activity.

In some aspects, the techniques described herein relate to a prosthetic or orthotic device, wherein a first entry condition of the one or more entry conditions corresponds to a torque value satisfying a torque threshold, wherein the controller is further configured to: based at least in part on a determination that the first entry condition was satisfied, select the sit-to-stand transition.

In some aspects, the techniques described herein relate to a prosthetic or orthotic device, wherein the sit-to-stand transition, has at least two subphases, wherein each of the subphases corresponds to different actuator behaviors.

In some aspects, the techniques described herein relate to a prosthetic or orthotic device, wherein the prosthetic or orthotic device further includes of one or more position sensors.

In some aspects, the techniques described herein relate to a prosthetic or orthotic device, wherein a first entry condition of the one or more entry conditions corresponds to an angle between the first limb member and the second limb member satisfying an angle parameter threshold, wherein the controller is further configured to: based at least in part on a determination that the first entry condition was satisfied, select a sit-to-stand transition.

In some aspects, the techniques described herein relate to a prosthetic or orthotic device, wherein the gait activities include of at least a slow walk gait activity and a fast walk gait activity.

In some aspects, the techniques described herein relate to a prosthetic or orthotic device, wherein the fast walk gait activity corresponds to a first set of actuator behaviors during a stance phase, and a second set of actuator behaviors during a swing phase.

In some aspects, the techniques described herein relate to a prosthetic or orthotic device, wherein the controller may use the slow walk gait activity as an intermediate gait activity between two other gait activities.

In some aspects, the techniques described herein relate to a prosthetic or orthotic device, wherein at least one of the gait activities has at least two subphases, wherein each of the subphases corresponds to different actuator behaviors.

In some aspects, the techniques described herein relate to a prosthetic or orthotic device, wherein the gait activities include of at least a yield gait activity.

In some aspects, the techniques described herein relate to a prosthetic or orthotic device, wherein the controller uses the yield gait activity as an intermediate gait activity between two gait activities.

In some aspects, the techniques described herein relate to a prosthetic or orthotic device including: a first limb member; a second limb member coupled to the first limb member at a joint; an actuator coupled to the first limb member and the second limb member and configured to actuate the first limb member relative to the second limb member; a torque sensor; and a controller configured to control the actuator via a control map, wherein the control map includes of gait activities and gait activity transitions, wherein each of the gait activities and the gait activity transitions correspond to one or more actuator behaviors, wherein the gait activities include of at least a slow walk gait activity and a fast walk gait activity, and wherein the fast walk gait activity corresponds to a first set of one or more actuator behaviors during a stance phase, and a second set of one or more actuator behaviors during a swing phase, and wherein the controller is further configured to: based at least in part on a determine that one or more entry conditions of a gait activity are satisfied, select a gait activity transition to transition from a first gait activity to a second gait activity, wherein the second gait activity is determined based on the one or more entry conditions that were satisfied.

In some aspects, the techniques described herein relate to a prosthetic or orthotic device, wherein the first set of one or more actuator behaviors corresponding to the fast walk gait activity include of at least a force rejection behavior and a toe-off assist behavior.

In some aspects, the techniques described herein relate to a prosthetic or orthotic device, wherein the second set of one or more actuator behaviors corresponding to the fast walk gait activity include of at least a force following behavior and a breaking behavior.

In some aspects, the techniques described herein relate to a prosthetic or orthotic device, wherein the second set of one or more actuator behaviors corresponding to the fast walk gait activity further include of at least a bumper avoidance behavior.

In some aspects, the techniques described herein relate to a prosthetic or orthotic device, wherein the prosthetic or orthotic device further includes of a ground contact sensor.

In some aspects, the techniques described herein relate to a prosthetic or orthotic device, wherein one of the gait activity transitions is a sit-to-stand transition, wherein the controller uses the sit-to-stand transition to transition from a sit gait activity to a stand gait activity.

In some aspects, the techniques described herein relate to a prosthetic or orthotic device, wherein a first entry condition of the one or more entry conditions corresponds to a torque value satisfying a torque threshold, wherein the controller is further configured to: based at least in part on a determination that the first entry condition was satisfied, select the sit-to-stand transition.

In some aspects, the techniques described herein relate to a prosthetic or orthotic device, wherein the sit-to-stand transition, has at least two subphases, wherein each of the subphases corresponds to different actuator behaviors.

In some aspects, the techniques described herein relate to a prosthetic or orthotic device, wherein the prosthetic or orthotic device further includes of one or more position sensors.

In some aspects, the techniques described herein relate to a prosthetic or orthotic device, wherein a first entry condition of the one or more entry conditions corresponds to an angle between the first limb member and the second limb member satisfying an angle parameter threshold, wherein the controller is further configured to: based at least in part on a determination that the first entry condition was satisfied, select a sit-to-stand transition.

In some aspects, the techniques described herein relate to a prosthetic or orthotic device, wherein the controller may use the slow walk gait activity as an intermediate gait activity between two other gait activities.

In some aspects, the techniques described herein relate to a prosthetic or orthotic device, wherein at least one of the gait activities has at least two subphases, wherein each of the subphases corresponds to different actuator behaviors.

In some aspects, the techniques described herein relate to a prosthetic or orthotic device, wherein the gait activities include of at least a yield gait activity.

In some aspects, the techniques described herein relate to a prosthetic or orthotic device, wherein the controller uses the yield gait activity as an intermediate gait activity between two gait activities.

### BRIEF DESCRIPTION OF THE DRAWINGS

FIG. 1A illustrates one embodiment of a powered knee prosthesis.

FIG. 1B illustrates another embodiment of a powered knee prosthesis.

FIG. 2 is a block diagram of the main hardware components of a powered prosthetic knee device.

FIG. 3 is a schematic representation of a controlled system architecture for a prosthetic or orthotic device.

FIG. 4 is a block diagram of an impedance controller used to control an actuator of a powered knee prosthetic.

FIG. 5 is a high-level block diagram of a control map illustrating the locomotion activities and activities transitions of an embodiment of the control system implemented on a powered prosthetic knee.

FIG. 6 is a diagram illustrating the basic phases and subphases associated with the sit-to-stand transfer activity and neighboring activities.

FIG. 7 is a schematic of an extension (EXT) subphase actuator behavior management process.

FIG. 8 is a diagram illustrating the fast walk gait activity management strategy.

FIG. 9 is a fast walk gait cycle knee joint angle curve with the matching phases and subphases showing fast walk gait cycle characteristics.

FIG. 10 is a fast walk gait cycle knee joint torque curve with the matching phases and subphases showing fast walk gait cycle swing phase torque.

FIG. 11 is a high-level block diagram of a control map illustrating the locomotion activities and transitions of an embodiment of a control system for a powered knee prosthetic device where the SDN gait activity is managed separately from yield gait activity.

### DETAILED DESCRIPTION

Current actuator control strategies deployed for passive lower-limb prosthetic joints are directed to systems where the energy is observed to flow in a single direction, from the user generated load and efforts towards the device which acts to dissipate the energy. In contrast, when dealing with a device capable of generating net positive mechanical power energy flows in both direction between the user and the device. In power generating prosthetics, the energy exchange between the user and the device is also observed to dynamically change over time. Additionally, the energy exchange may not be fully consistent between occurrences of the same gait activities or cycles since energy generation is strongly influenced by the environment in which the task is being executed and the constraints affecting the user executing the gait task.

Use of general regulation strategies that may perform adequately when dealing with an actuator that is limited to dissipating energy are not well suited to use with power generating actuators. While they may provide adequate performance in some specific situations their use cannot be generalized. A more generalized control approach that allows control systems to address the dynamic changes in the energy flow direction between the user and device may be used.

General regulation strategies may also perform poorly because the motion control used in passive prosthetics cannot be executed in a feedforward manner for long periods of time while maintaining high usability and clinical performance. In themselves, the locomotor and non-locomotor task execution observed on the human lower limb present a high variability to be addressed with some form of arc-reflex loop. The arc-reflex loop allows a person to smooth out the impact of the external factor on the task execution. A multitude of factors can affect the gait execution at any point of the gait cycle. To properly enforce the clinical function and objectives, a control system allowing reactive behavior to take place is desired and is not currently found to be available.

The embodiments herein disclosed provide benefits over the current state-of-the-art by allowing a control system to be reactive and adaptable, instead of a discrete state high-level control system. By allowing the low-level control architecture components the flexibility to manage common reactive actuator behaviors, without requiring explicit change of the high-level states management, the system can dynamically manage the varying energy flow between the user and the device. Additionally, the system can manage the energy flows inside of specific gait activity and/or states. In certain cases, the system may manage energy flows to optimize system usability from the user perspective.

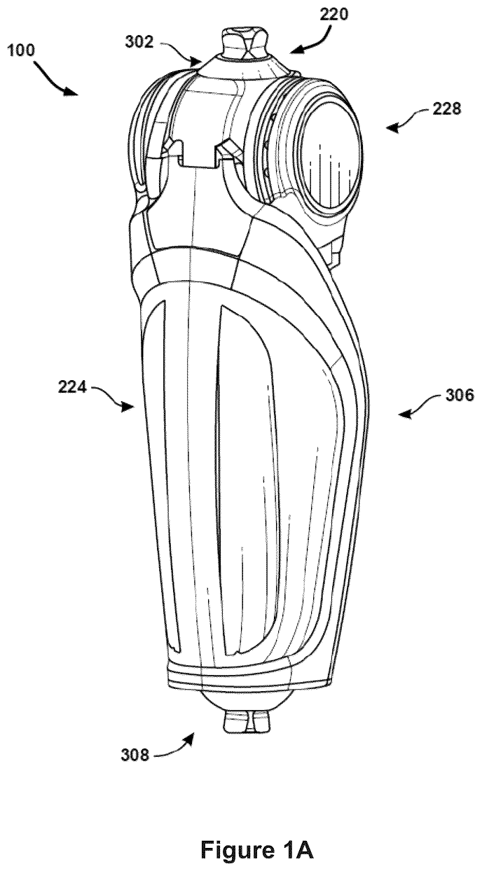

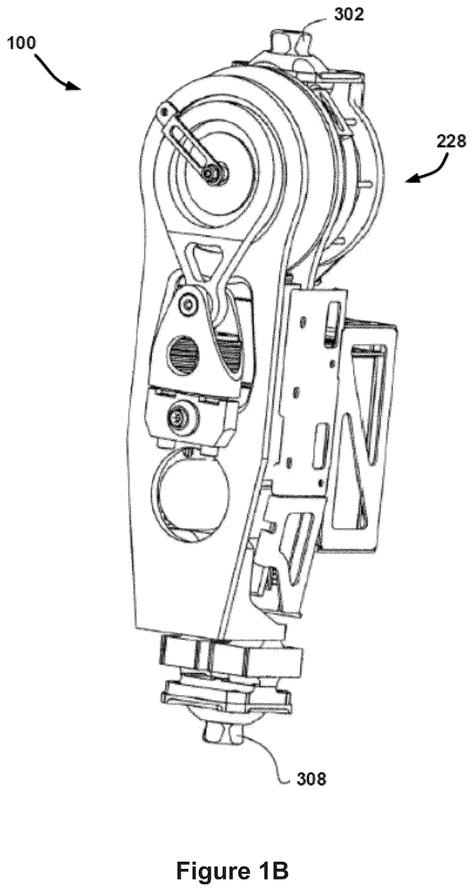

FIGS. 1A and 1B illustrate examples of prosthetic or orthotic devices (PODs) 100. The PODs 100 illustrated in FIGS. 1A and 1B implement a knee joint and may be connected to a user's residual limb, such as a thigh or a stump of an above-knee amputee, through a socket (not shown) via the proximal connector 302 on its proximal segment 220. The proximal connector 302 may be pyramidal, rectangular, or cylindrical in shape. In the examples of FIGS. 1A and 1B, the POD 100 is a lower-limb prosthetic device comprising structures to replace the anatomical structure of the human knee and upper part of the shank. The shank may include the anatomical structure below the knee and above the ankle.

The proximal connector 302 may be coupled to an actuator 228, which can rotate with respect to a body 306. The actuator 228 may be motorized. Rotation of the actuator 228 may cause rotation of the proximal connector 302 with respect to the body 306, and vice versa. In some aspects, the body 306 may be on or form a shank portion of the POD 100. The body 306 may include or house electronic components, sensors, etc. (not shown) or other components used to operate the POD 100, although in some aspects these components may be located elsewhere, such as on a peripheral device or within remote components. In some cases, the POD 100 may be connected to a prosthetic or orthotic ankle or foot (not shown) via the distal connector 308 located on a distal segment 224 of the POD 100. The POD 100 may be connected with or include a prosthetic hip, prosthetic thigh, prosthetic foot, prosthetic ankle, or the like.

Example PODs are described in U.S. Pub. No. 2009/0299480, filed Jul. 7, 2009, entitled “Joint Actuation Mechanism for a Prosthetic or Orthotic Device Having a Compliant Transmission,” U.S. Pub. No. 2011/0125290, filed Feb. 2, 2011, entitled “Reactive Layer Control System For Prosthetic And Orthotic Devices,” each of which describes various embodiments and features related to POD systems and each of which is hereby incorporated herein by reference in its entirety.

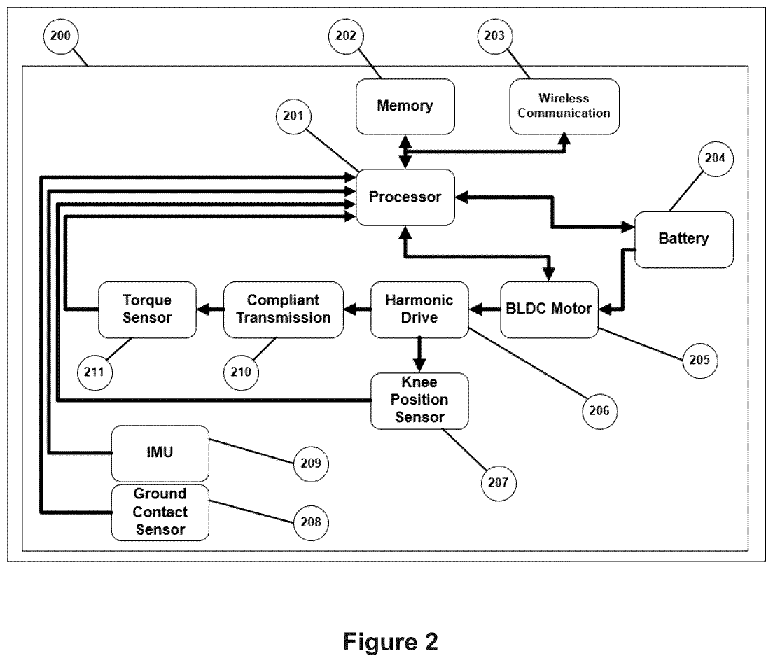

FIG. 2 illustrates a block diagram 200 of example hardware components (e.g., the building blocks) for a processor-controlled powered prosthetic knee 100, such as the examples provided in FIGS. 1A and 1B. The POD 100 may use a brushless DC motor 205 combined with a harmonic drive 206 and a compliant transmission element 210. Alternatively, or in addition, the POD 100 may use one or more AC motors and/or brushed motors. The processor 201 may connect most of the other building blocks together. The processor 201 may operate in such a way as to create a cohesive ensemble from the individual components and modules. The processor 201 is in operational communication with a memory 202, where information for the correct system operation can be stored and retrieved. Additionally, or alternatively, the processor 201 manages the wireless communication module 203 to exchange information with external devices, such as mobile phones or tablets, where various types of applications can be used to retrieve information from the POD 100, trigger specific functions, or provide new data to be stored in the memory 202.

The POD 100 may include a battery 204 to power the electronic components, such as the various sensors 207, 208, 209 and 211, the processor 201, and the motor 205. Various types of battery technologies can be used to power the control system of the POD 100. However, in some embodiments, the mobile nature of the system may limit the weight of a battery or power system. Thus, battery technologies providing high power and energy densities may be better suited for this type of application. Additionally, the POD 100 may use secondary type battery technologies to reduce the operating costs of these devices by allowing the battery to be recharged, as well as reduce the environmental impact related to the device's operation.

Embedded sensors are integrated in the basic components of the system hardware. The embedded sensors may be used as input sources for implementing the various control loops and intent management functions. In some embodiments, the POD 100 may use a ground contact sensor 208 to monitor the interaction between the user wearing the prosthetic or orthotic device 100 and the immediate environment. Various constructions can be adopted to implement the ground contact sensor 208, such as, but not limited to, loadcells, optical displacement sensors, strain gauges, magnetic displacement sensors, inductive displacement sensors, capacitive displacement sensors, pressure sensors, and piezoelectric load sensors. In one non-limitative embodiment of a ground contact sensor system, the ground contact sensor array disclosed in U.S. Provisional Application No. 62/894,442 filed Aug. 30, 2019 and U.S. Nonprovisional application Ser. No. 17/638,493, which was filed Feb. 25, 2022, can be used to provide sensing capabilities to segregate between heel load and toe load due to sensor mechanical properties and use of a sensor array, which is hereby incorporated by reference in its entirety.

In some embodiments, a load sensor assembly may be located at a distal end of the prosthetic device 100 near the distal connector 308. The load sensor assembly may have one or more load sensors (e.g., one load sensor, five load sensors, seven load sensors, more than seven load sensors, or any number in between)). The locations of the sensors may represent different load-bearing areas of the prosthetic device 100. For example, the load-bearing surface may be a bottom of a prosthetic or orthotic foot or an ankle. For example, the POD 100 may have two posterior load sensors located towards the rear of the assembly that represent the posterior portion/area of a prosthetic or orthotic foot and have two anterior load sensors located towards the front of the assembly that represent the anterior portion/area of the prosthetic or orthotic foot. More specifically, the two posterior load sensors may represent the heel of the prosthetic or orthotic foot, while the two anterior load sensors may represent the toes of the prosthetic or orthotic foot. In some embodiments, the POD 100 may have three mid-section sensors that represent the mid-section of the prosthetic or orthotic foot. Likewise, the sensor assembly may have one or more sensors that represent the medial (or lateral) side of a prosthetic or orthotic foot. It should be understood that any number of sensors (e.g., one sensor, two sensors, three sensors, more than three sensors, or any number in between) may be used to represent different load-bearing areas, and that some of the load sensors may represent multiple load-bearing areas (e.g., the posterior-lateral area/portion of a prosthetic or orthotic foot).

The POD 100 may integrate one or more Inertial Measurement Units (IMU) 209 to measure the kinematics of the user's residual limb or the prosthetic device. In some embodiments, the POD 100 may include a knee position sensor 207 or another type of sensor that can be used to measure knee joint kinematics directly, which are the result of the interaction between the user, the environment, and the POD's 100 actuator. In some embodiments, the POD 100 may have a knee angle sensor (e.g., encoder).

In some embodiments, the POD's 100 actuator may include the brushless DC motor 205, the harmonic drive 206 transmission and the compliant transmission element 210. The POD's 100 actuator may alternatively be separate from and work together with the brushless DC motor 205, the harmonic drive 206 transmission and the compliant transmission element 210 to control the POD 100.

In some embodiments, the POD 100 may include a torque sensor 211 that may directly measure the interactions occurring between the prosthetic device and the user, providing a direct measure of the system kinetics. The sensor described above may be functionally connected with the processor 201. The processor 201 may retrieve the information from the connected sensors and further process it based on the definition of the detection and control processes.

In some embodiments, the brushless DC motor 205 is functionally connected to and controlled by the processor 201 based on the outcome of the data processing performed in firmware. As mentioned above, the motor-transmission combination, or any other type of actuator, may implement the application-level strategies through motion control of the actuator. Implementation of the motion control strategies, combined with environmental and user interactions may cause a general system response that can be measured by the sensors, hence closing the feedback control loop.

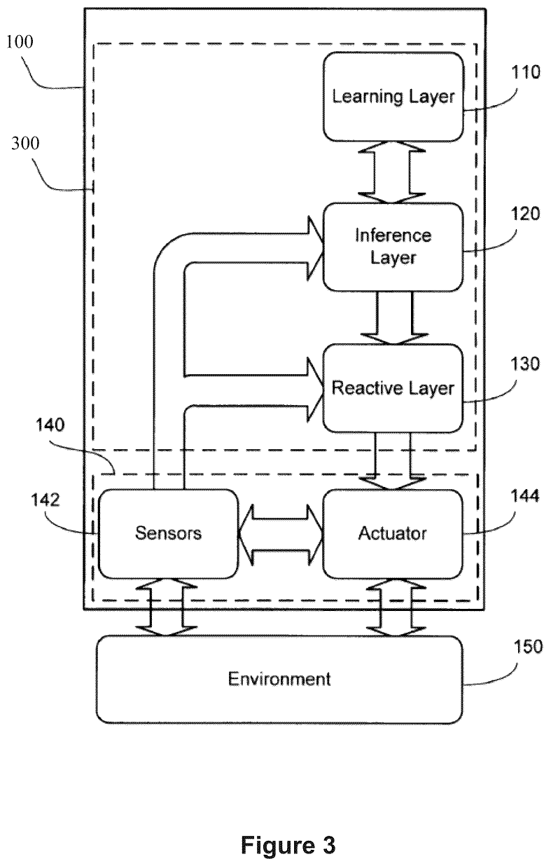

The processor 201 can be leveraged to implement a layered control system. Use of a layered control system architecture for the implementation of the application-level control mechanisms advantageously allows for simple and efficient organization of the data flow in the control system, as well as the creation of a data abstraction model that efficiently divides the tasks between the modules that are cohabiting in the device firmware. An example of such layered control system is presented in FIG. 3.

FIG. 3 illustrates one embodiment of the organization of the control system of a POD 100. This organization of software processes, data streams, and a priori knowledge is referred to herein as a multi-layer hierarchical architecture. This method of architectural organization can satisfy at least two objectives, namely provide high performance control of an active POD, and efficiently organize processes and data stream(s) such that information received from the POD's 100 sensors propagates through the system in an orderly and logical fashion.

In some embodiments, the control system of the POD 100 may be or may include a multi-layered controller 300 with three or more layers. For example, the learning layer 110, the inference layer 120, and the reactive layer 130 in FIG. 3 depict the hierarchical layering of the control system architecture of an active POD 100. Moreover, the motorized prosthetic or orthotic apparatus 100 and environment 150 highlight the interactions between the control system and the environment in which the POD 100 operates. In some embodiments, the motorized prosthetic or orthotic apparatus 100 includes external interfaces 140 that may be connected to the control system. In some embodiments, the external interfaces 140 may include one or more sensors 142 and/or one or more actuators 144.

In certain embodiments, the control system is organized into hierarchical layers based on an examination of the flow of data into and out of the layers, as well as the interdependencies between layers. The layer names, data abstraction models, and nature of the data stream associated with certain embodiments are described herein using physiological terms. The three layers that can be used to sustain active POD 100 operation in certain embodiments include, but are not limited to, a learning layer 110, an inference layer 120, and a reactive layer 130. The different layers can be categorized based on the level of abstraction, the time frame in which they act, etc.

The various layers interact with each other to improve the performance of the POD 100. For example, a well-tuned POD may provide reasonable performance using (only) the reactive layer 130 and inference layers 120 but may be unable to evolve to meet the user's changing expectations or respond to long term changes in the operating environment without the use of a learning layer 110. In some embodiments, the users of the POD 100 may include an end-user (e.g., an amputee), a clinician or clinical staff member, and/or a manufacturer or manufacturing staff member.

The learning layer 110 can include the control structure's highest abstraction level and can be the level furthest away from raw sensor data. The learning layer 110 can be loosely analogized to human cognitive functions, and it can be used to recursively improve the POD control system's performance as time passes. In addition, the learning layer 110 can have the longest time frame in which it acts. For example, in certain embodiments, the learning layer 110 does not respond to changes in gait pattern between steps. Rather, the learning layer 110 identifies and responds to long-term trends in user performance.

The learning layer 110 can also, or alternatively, define what is considered an improvement in performance. The criteria for improved or reduced performance can evolve with time as the user becomes more familiar with the POD 100 and demands a higher level of performance. In some embodiments, this evolution may be thought of as the POD's transition from a POD possessing a moderate level of performance coupled with a very high level of safety to a POD possessing a high level of performance coupled with reduced user safety constraints, thereby increasing the POD's flexibility and performance potential. As it evolves, the learning layer 110 can also decide to allow user access to features that had previously been hidden or were not made available. In some embodiments, the user may select different criteria for the learning layer 110 to prioritize. For example, the user may instruct the learning layer 110 to prioritize safety, performance, and/or battery life.

In certain embodiments, the learning layer 110 can include an expert system consisting of rule and data sets that can make decisions as to how well the POD performs in particular situations (e.g., walking, climbing steps, standing up, etc.) and environments (e.g., inside, outside, rainy, etc.) and use the inference layer 120 to implement the high-level decision making. The learning layer 110 alters the rule set or parameter values used by the inference layer 120 incrementally and over longer periods of time to dictate the POD's 100 performance.

In addition, the learning layer 110 can provide support as the controller formulation itself may sometimes change. For example, a single actuated POD 100 can potentially cover a large and diversified range of locomotion activities and the user can progressively adapt its behavior, which may rely on additional adjustment to enable the system to maintain a stable level of performance.

The inference layer 120 can be responsible for a variety of different activities. These activities can include, but are not limited to, identifying the current activity or gait phase being performed by the user, predicting future activity or gait phase performed by the user, measuring the performance of the POD 100, requesting that the learning layer 110 examine a particular performance issue, organizing and passing the requested/required data to the learning layer 110, providing the reactive layer 130 with enough data to smoothly execute the task at hand, determining the basic phase of the POD 100 (for example, standing, walking, etc.), determining whether to select active mode or passive mode, etc.

Within the control system, the inference layer's 120 role can be conceived of as mimicking the human brain's conscious decision-making process. Using the rules and data it acquires from the learning layer 110, the inference layer 120 can infer which course of action is appropriate from sensory data measurements and estimates. Thus, the inference layer 120 can mimic the human ability to apply what one has learned to specific present and future situations.

Unlike the learning layer 110, the inference layer 120 reacts over the course of a single gait cycle. The inference layer 120 can quickly apply rules it has learned from the learning layer 110 to situations as they arise and respond accordingly. For example, the response time of the inference layer 120 can be on the order of tenths of seconds. In some embodiments, the inference layer 120 apply rules over multiple gait cycles. For example, the inference layer 120 can modify the operation of the POD 100 if it detects a change in environment or condition of the user (e.g., fatigue, injury, etc.).

Contextually, the inference layer 120 can be thought of as an intermediate data abstraction level, where the work includes extracting features from an input stream comprising pre-processed data. The data can be processed and characterized to achieve objectives including, but not limited to, managing the POD 100 such that system behavior matches the activity being undertaken by the user and quantifying system performance in terms of overall functionality of the POD 100.

The intermediate position the inference layer 120 occupies within the hierarchy can serve an additional purpose: protecting the reactive layer 130 from the learning layer 110. That is, the inference layer 120 can prevent the learning layer 110 from changing parameters directly used by the reactive layer 130, which may lead to system instability. Instead, the learning layer 110 can direct the inference layer 120 to take a particular action or slightly alter a variable in a data set.

The reactive layer 130, in certain embodiments, represents the lowest abstraction level of the multi-layered controller 300. The reactive layer 130 can directly enforce the desired behavior that is responsive to the activities undertaken by the user and the current environment of the user. Here, the desired behavior can be determined by the inference layer's 120 rule set, which the learning layer 110 can alter. In other words, similar to the general behavior of the human arc-reflex, the reactive layer 130 of certain embodiments can immediately enforce a predefined behavior based on a reduced set of sensory input and estimates. The time frame for the operation of the reactive layer 130 can be on the order of milliseconds or less. More specifically, the reactive layer 130 can handle the motor control laws of the POD 100.

Although the control system architecture presented in FIG. 3 may appear to be hierarchical in nature, the control system architecture can be parallel in structure. For example, the control system may direct the reactive layer 130 to manage itself. For example, in certain embodiments, the reactive layer 130 can use a knee torque sensor output to implement a low-level impedance controller to manage the POD's 100 actuator behavior in real-time. Alternatively, or in addition, the same knee torque sensor output can be used by the inference layer 120 to detect the occurrence of a transition in the user's gait activity to downwards walking. Both decisions present different levels of complexity that can be made independently by the different control layers of the multi-layered controller 300.

Additional details regarding control layers useable with certain embodiments of the invention are disclosed in U.S. Patent Publication No. 2011/0125290 (the “'290 publication”), published May 26, 2011, entitled “Reactive Layer Control System for Prosthetic and Orthotic Devices,” which describes various embodiments and features related to POD systems and which is hereby incorporated herein by reference in its entirety.

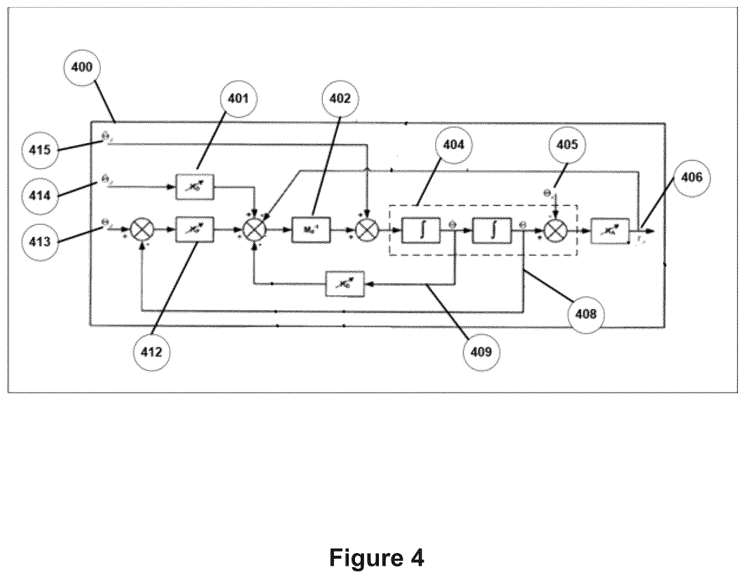

FIG. 4 illustrates a block diagram of one embodiment of an impedance controller 400 of the POD 100. The impedance controller 400 can be implemented at the reactive layer 130. The impedance controller 400 allows the POD 100 to manage the actuator 228 or support the implementation of various behaviors by the actuator 228. For example, the impedance controller 400 may determine the torque to be generated by the actuator to create a predetermined dynamic response or behavior. In some embodiments, the predetermined dynamic actuator response can be determined based on the gait activity, phase and subphase information at the inference layer 120 level.

The impedance controller 400 may be formulated around the combined actuator and knee prosthesis dynamic model 404, to which an external input 405 is used to represent the user interaction with the physical device, represented as a torque or position perturbation added to actuator and prosthetic system dynamic response. In a general and non-limitative manner, the powered prosthetic device is represented as a double integrator. Full implementation of the impedance controller 400 makes use of position feedback 408, velocity feedback 409, and/or force feedback signal 406. Feedback signals can be measured using physical sensors (e.g., encoders, load cells, torque sensors, IMUs, etc.), or estimated based on observation of other physical quantities (e.g., motor current for torque, output position for velocity, etc.).

Feedback signals 406, 408 and 409 are compared to the setpoint signals for position 413, velocity 414 and force/acceleration 415 as part of the feedback controller formulation. While the gains can define the dynamic response of the controlled system, the setpoint signals may define a kinematic or kinetic trajectory for the system to follow, additionally to the dynamic response. Time-varying or constant set-points can be used, creating either a tracking controller or a regulator, accordingly to the specific control problem at hand. Additionally, time-varying or constant gains can be used.

In the context of use related to a powered prosthetic device actuator control, a regulator formulation of the control loop is advantageously used for energy dissipative tasks. For example, regulating the knee velocity while yielding under the user weight in stance phase of stairs descent. A tracking controller implementation where the set-point is dynamically varied is advantageously used in power generative tasks, where the knee joint is to be changed angle under the user's weight. For example, when the user is performing the sit-to-stand transition and the knee joint is to extend under the user's weight.

Due to the variable nature of the knee's role across the gait activities, phases and subphases, it is advantageous for the control system of the POD 100 to change the knee joint actuator behavior accordingly. The capacity to dynamically adapt the impedance controller gains based on the phase, subphases and gait activity can be advantageously leveraged to create a simple mechanism to adapt the actuator behavior while maintaining a single controller implementation.

It should be understood that while it can be beneficial to use all the gains and/or set-points signals introduced above simultaneously, in many cases the gait tasks requirements will not require the use of all terms or features of the impedance controller 400. Since the impedance controller 400 combines three simpler control loops, independent use of any of these control loop or any combination of these is possible and desirable in some circumstances.

In some cases, the POD 100 may use the impedance controller 400 to define the actuator dynamic behavior as passive. For example, based on the inference layer 120 determining that the user of the POD 100 is standing, a command can be issued to the reactive layer 130 to enter a passive mode. During the passive mode, the actuator can exhibit a force rejection (FR) behavior during some of or the entire stance phase and a force following (FF) behavior during some of or the entire swing phase. The FR and/or FF behaviors may be implemented by defining the impedance controller position 412, velocity 401 and mass 402 gains the appropriate values. These gains can be empirically tuned through series of experiments or calculated based on the desired dynamic response and the prosthetic system characteristics.

In some cases, the POD 100 may use the impedance controller 400 to define the actuator behavior as active. For example, based on the inference layer 120 determining that the user of the POD 100 is walking, the inference layer 120 can communicate a command to a reactive layer 130 to enter an active mode. During the active mode, the impedance controller 400 can cause the actuator to exhibit a FR behavior and a toe-off assist (TOA) during the stance phase. For example, during the active mode, the actuator can exhibit the FR behavior between heel-strike and midstance and the TOA between midstance and toe-off. The FR and TOA behavior can be defined through definition of the appropriate gain and set-point values. As described above, the gain and set-point values can be empirically tuned through series of experiments or calculated based on the desired dynamic response and the prosthetic system characteristics. In some embodiments, the learning layer 110 can adjust the gain and set-point values to improve the performance of the FR, FF, and/or TOA behaviors.

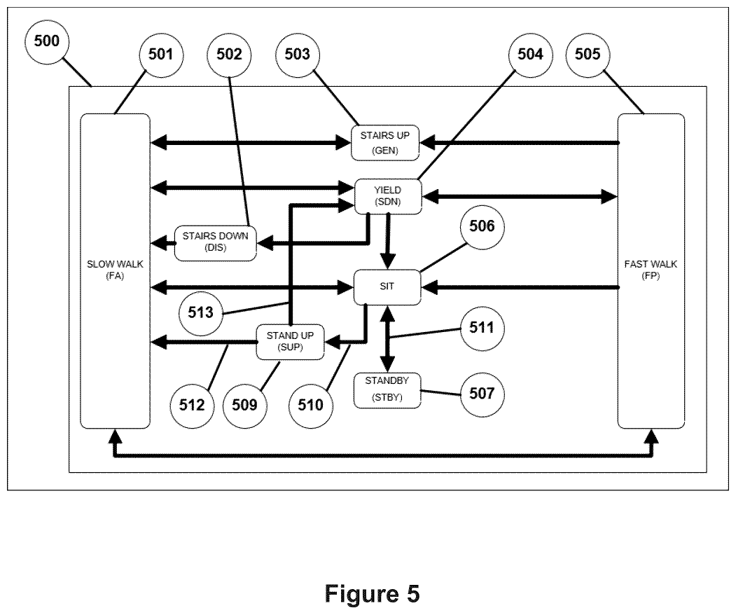

FIG. 5 presents a high-level block diagram of an embodiment of the control map 500 illustrating various gait activities and transitions that can be advantageously implemented by the control system. In view of the clinical application and functional features to be deployed to solution the clinical application problem, various configuration of the activities to be supported by the POD 100, as well as supported transitions between said activities, various configurations of the activity definition, and control map can be created without departing from the baseline intent of the embodiments herein disclosed.

In control map 500 (e.g., the gait activities and transitions map), eight activities are used to cover the gait activities associated with the clinical application of the POD 100. The activities represent common locomotor and non-locomotor gait activities observed in daily living. Namely, slow walk (FA) 501, fast walk (FP) 505, stairs up (GEN) 503, yield (SDN) 504, sit 506, standby (STBY) 507, stand-up (SUP) 509, and stairs down (DIS) 502 are the gait activities implemented as part of the control system. In some embodiments, each gait activity and gait activity transition may correspond to one or more actuator behavior. Based on the general layering used in the type of control system presented in POD 100, these activities and transitions may be defined and managed at the Inference Layer 120 level. For example, each gait activity and transition may have one or more entry conditions and/or one or more exit conditions. The entry and exit conditions, as well as the corresponding actuator behaviors of the gait activities and transitions will be described in more detail below. Though the control map 500 shows eight activities, one of skill in the art will recognize that some embodiments of the control map may have more activities or less activities, such as nine, ten, or more than ten activities.

For example, in some embodiments, the control map includes a kneeling gait activity and/or error gait activity. In some embodiments, the kneeling gait activity sets the POD 100 to passive mode (e.g., POD 100 does not provide net positive power). In some embodiments, the kneeling activity is triggered when the POD 100 is horizontal or sufficiently rotated out of the sagittal plane for a (minimum) duration (100 ms, 200 ms, 500 ms, more than 500 ms, or any amount of time in-between). While in the kneeling gait activity, the POD's 100 knee may be passive/compliant (e.g., in transparent mode). The POD 100 may transition to the standby 507 gait activity if the POD 100 is stationary while in the kneeling gait activity for a duration threshold.

In some embodiments, the error gait activity is triggered if an error is detected on a component (e.g., an angle sensor, current sensor, ground contact sensor 208). In some embodiments, POD 100 will extend with fixed current on the motor during the error gait activity. The error gait activity may be used to prevent the POD from being used when the defined control methods are unavailable. The amount of current used to extend the POD 100 in the error gait activity mode is sufficient to extend the knee while not preventing a user from overpower the knee if he/she wants to. In some embodiments, the error gait activity may not be a normally accessible activity but rather a risk management mode and a safety mechanism.

The sit 506 activity corresponds to the gait activity when the user is operating the lower-limb prosthetic device while seated. This non-locomotor gait activity allows the control system to manage the prosthetic device behavior such that the user capacity can comfortably sit and not be restricted in his or her movements. In some embodiments, during the sit 506 activity, the control system modifies the POD's 100 behavior based at least in part on the specific dimension and configuration of the seat which is used. For example, the POD 100 may detect the dimension and configuration of the seat via one or more sensors disclosed herein and/or past user data. In some embodiments, the POD 100 operates in the FF mode during the sit 506 activity.

The sit gait 506 activity can be entered in several manners, which allows greater flexibility to account for the various strategies adopted by the users. Failure to properly account for the variability in the strategies adopted to reach the seated position may result in reduced device usability or prevent proper assistance to be delivered to the user. Typical activity flow leading to a sit gait activity 506 detection would normally transition through slow walk 501 when the user stops and stands in front of or next to a seat and yield 504 when the stand-to-sit transition is initiated. Some users may go directly from slow walk 501 or fast walk 505 to sit 506 (e.g., by sitting down using the non-amputated leg to manage the transition potentially due to a desire to be seated faster or from bad gait habits developed using lower limb devices that did not properly support the stand-to-sit transfer). The sit gait activity 506 can be entered from the standby 507 activity.

The POD 100 can detect which transition is occurring and adapt the behavior of the POD 100 accordingly to improve performance, user safety, and user comfort. While entering from slow walk 501, fast walk 505 or yield 504, the sit 506 activity transition may rely on detecting the thigh segment being generally vertical and the phase being observed as swing. In the case where sit 506 is entered from the standby 507 gait activity, detection of the gait activity change by the control system inference layer 120 may rely on detecting motion of the prosthetic device through the knee joint angle sensor and/or inertial measurement unit. In some embodiments, the knee joint angle sensor determines the angle between a first limb member and a second limb member. For example, the first limb member may be and/or include the proximal connector 302 and/or the proximal segment 220 and the second limb member may be and/or include the body 306, distal segment 224, and/or distal connector 308 of the POD 100. In some cases, the first limb member is colinear with the user's thigh or residual limb. For example, the first limb member may connect to a socket in which the user's residual limb (e.g., thigh) is placed.

In some embodiments, a relative knee angle of zero may correspond to a neutral position of the knee (e.g., the first limb member and the second limb member being generally colinear with each other). The relative knee angle may increase with knee flexion and may decrease with knee extension.

Furthermore, in some embodiments, the IMU or another sensor may measure the absolute angle of the second limb member. The absolute angle of the second limb member may be relative to an external reference plane (e.g., the ground plane or a horizontal plane). In some embodiments, an absolute angle of zero may correspond to the second limb being in a vertical or neutral position (e.g., a standing position). In some cases, a positive absolute angle may correspond to a second limb that is positioned such that the top of the second limb is positioned further forward than the bottom of the second limb relative to the center-of-gravity of the user (e.g., position of second limb during knee flexion). A negative absolute angle may correspond to a second limb that is positioned such that the top of the second limb is positioned further back than the bottom of the second limb (e.g., when in a sitting position with the second limb extended out away from the body). A similar coordinate system may be used to describe the movement of the first limb member in the sagittal plane. For example, a vertical position may be zero degrees, hip flexion may increase the absolute angle (e.g., top of thigh is posterior to bottom of thigh), and hip extension may decrease the absolute angle (e.g., top of thigh is anterior to bottom of thigh).

In some embodiments, the sit 506 activity can be exited in at least three different ways, which may cover the main functional use cases associated with the activity of daily living. In the case where the user is observed to be sitting for a predetermined amount of time without significant motion being registered by the sensor system, POD 100 transitions to standby 507 gait activity. For example, during a stance transition where (1) the knee is generally extended (e.g., the relative knee angle is less than approximately 10 degrees) and (2) the first limb member becomes vertical, the POD 100 may transition directly to the slow walk 501 gait activity from the sit 506 activity. This scenario may be observed when the user stands up from the seated position using the sound limb. Once the standing position is reached, the stance phase detection may determine that the prosthetic limb is loaded. The determination that the POD 100 is loaded (e.g., supporting at least some of the user's weight) may cause the POD 100 to transition to slow walk 501 gait activity. In some embodiments, if the control system detects knee extension (e.g., relative knee angle decrease) and thigh segment roll-over, the control system may transition to the stand-up 509 gait activity. The control system may detect the thigh segment roll-over when the hip transitions from hip flexion to hip extension.

In some embodiments, the POD 100 may determine the position and/or movement of a user's hip and/or residual limb on the sagittal plane by comparing the position of the second limb member with the relative knee joint angle. As described above, the POD 100 may use the knee joint sensor to measure the knee joint angle and the IMU to measure the position or absolute angle of the second limb. In some embodiments, the POD 100 may determine that the user's hip is extending based at least in part on a determination that the absolute angle of the shank is not changing, or changing less than a threshold amount (e.g., less than 5 degrees), and the relative angle of the knee joint is decreasing. Similarly, the POD 100 may determine that the users' hip is flexing based at least in part on a determination that the absolute angle of the shank is not changing, or changing less than a threshold amount (e.g., less than 5 degrees), and the relative angle of the knee joint is increasing.

In some cases, the POD 100 may determine the position and/or movement of a user's hip and/or residual limb by determining and comparing the position of the center of mass of the user's residual limb and second limb member. The POD 100 may use the knee torque measurement to determine the center of mass of the user's residual limb. For example, a larger knee torque may indicate that the center of mass of the user's residual limb is behind the center of mass of the second limb. Furthermore, a low or zero knee torque may indicate that the center of mass of the user's residual limb and second limb member are colinear (e.g., the user is in a neutral position).

The comparison described above may also, or alternatively, be used to determine the general posture of the user (e.g., the position of the user's upper body). For example, if the knee torque increases while the second limb remains stationary, the POD 100 may determine that the user's center of mass has moved (e.g., the user has leaned back). In some embodiments, the POD 100 may use (1) the change in relative knee angle relative to the absolute angle of the first limb and/or (2) the change or lack of change in the knee torque to determine the posture of the user (e.g., the position of the user's hip, thigh, etc.).

The standby 507 gait activity may define a system state in which no activities are expected for a time period. The time period may be 0.5 seconds, one second, two seconds, five seconds, longer than five seconds, or any amount of time in-between. When the POD is in the standby 507 activity state, various power saving actions may be enabled to increase and/or maximize device autonomy (e.g., battery life). For example, the motor 205, motor driver hardware, sensors, and/or other electronics may be disabled while the POD is in the standby 507 activity state, reducing system quiescent power usage.

In some embodiments, the standby 507 activity can be entered after a predetermined time period of no activity or low activity has been registered through the sensor set when operating in sit 506 gait activity through transition 511. The predetermined time period may be 0.5 seconds, one second, two seconds, five seconds, longer than five seconds, or any amount of time in-between.

The standby 507 activity may be exited through transition 511 back to sit 506 gait activity. In some embodiments, device modules or submodules may be brought back to full operation as part of a gait activity transition 511. The POD 100 may limit the number of scenarios to exit standby 507 gait activity to improve behavior consistency and system robustness.

The slow walk 501 activity defines a wide range of locomotion activities, which are characterized by the user standing on the lower limbs but not moving in such a way that could be characterized as cyclical walking. For example, during slow walk 501 activity the user can be standing still, shifting weight between the feet, moving about without reaching full cyclical lower limb behavior, or fully rolling over the support foot.

The slow walk 501 activity may, in some cases, be a default state for the control system. For example, upon power-up the slow walk 501 gait activity may be considered valid until further information about the system state and user action is available. The POD 100 may use the additional information to support system transition to another gait activity. Since slow walk 501 gait activity is used to support device operation when the user is standing or slightly moving, the slow walk 501 activity may be used in supporting transition between locomotor activities and can be entered from many supported gait activities. For example, slow walk 501 can be entered from the stairs up 503 gait activity upon detection that the stair ascent operation has been stopped for a (minimum) duration that is longer than a predetermined timeout condition. Additionally, dynamic transition from stairs up 503 to slow walk 501 gait activity can be executed upon detection of a sudden reduction of the cycle amplitude of the prosthetic limb's hip joint flexion, allowing the POD 100 to directly transfer from stairs ascent to level walking when the end of the staircase is reached. Similarly, slow walk 501 can be dynamically entered upon detection of a change in stance phase thigh segment roll-over when operating in stairs down 502 gait activity. Entering slow walk 501 when operating in yield 504 gait activity is also possible. For example, the transition may occur when the flexion torque level observed in ramp descent, fall below a minimum threshold. The reduction in flexion torque levels may indicate that the user has returned to a flat walking surface. As described above, the slow walk 501 gait activity may be entered when stand up 509 gait activity is complete and the user is standing on both limbs.

The POD 100 may also, or alternatively, transition from the sit 506 gait activity to the slow walk 501 gait activity. This transition may occur when the user stands up from a seated position without using the prosthetic limb to support the transition. As the knee passively extends under influence of gravity and reaches sufficient extension to allow stepping on it, the POD 100 may detect a stance phase with a mainly extended knee, which may cause the system to transition to slow walk 501 gait activity. Additionally, or alternatively, the slow walk 501 gait activity can be entered from the fast walk 505 gait activity when (1) the user's thigh or tibial progression drops below a predetermined threshold while in stance phase, (2) the walking cadence is observed to drop below a predefined threshold, (3) the elapsed time between two consecutive gait events is observed to exceed a maximum predetermined threshold, or (4) any combination of the conditions listed before.

The slow walk gait activity 501 may transition to many the POD's 100 supported locomotion activities, as well as the non-locomotor activities supported by the system. In some embodiments, slow walk 501 gait activity may be the used to support transitions between other type of gait activities. For example, the slow walk 501 activity may be an intermediate gait activity to connect the other gait activates together. In some embodiments, the slow walk 501 gait activity can be exited to stairs up 503 gait activity when the user begins climbing stairs from the standing position or when the user moves forward without reaching cyclical gait conditions. The POD 100 may transition from slow walk 501 to yield 504 when the user starts going down an inclined surface when previously operating in standing or non-cyclical walking. In some embodiments, the POD 100 transitions from the slow walk 501 activity to the sit 506 gait activity. For example, this transition may occur when the user performs the stand-to-sit transfer without using the prosthetic limb to control the lowering of his or her body's center-of-mass. The POD's 100 knee may operate in force following mode during the stand-to-sit transfer. When the POD's 100 is in FF mode the user can sit-down while manually flexing the knee joint. In some embodiments, the POD 100 transitions to the sit 506 gait activity when it detects that POD's 100 thigh segment is generally horizontal and/or that the POD's 100 shank is vertical with no load applied to the prosthetic foot. Additionally, or alternatively, the POD 100 may transition from the slow walk 501 gait activity to fast walk 505 activity if (1) the rotational velocity of the first limb member, the second limb member, and/or the knee joint increases above a predetermined threshold while in stance phase, (2) the user's walking cadence increases above a predefined threshold, (3) the elapsed time between two consecutive gait events (e.g., heel strike to heel strike or toe off to toe off) falls below a minimum timing threshold, or (4) any combination of the conditions listed before.

The stairs down 502 activity is used to manage the POD's 100 operation when the user performs a cyclical gait while going down stairs. This gait activity allows the POD 100 to control the knee flexion in stance phase such that the user's center-of-mass may be lowered in a controlled manner as the user progresses down one or more stairs. During swing phase the stairs down 502 activity uses powered extension of the POD 100 to reset the lower-limb position in view of the coming foot strike and weight transfer to the prosthetic limb. While managed in a similar manner as the yield 504 gait activity, the stairs down 502 gait activity presents slightly different dynamics to improve the operation of the POD 100. One example of such gait dynamics implementation is the swing phase extension. The stairs down operation may be characterized by a faster knee extension cycle in swing phase than in yield 504. Providing faster extension cycle velocity provides better support to the user while descending stairs and allows for more flexibility regarding cadence and the user's capacity to vary cadence.

In some embodiments, the stairs down 502 activity can be entered from the yield 504 gait activity. To improve system consistency and better address the user variability associated with the first step down a staircase being almost exclusively executed on the prosthetic limb, leading to a smaller step on the non-prosthetic limb, the first step when initiating ambulation down a staircase may be managed through the yield 504 gait activity. Through the yield 504 step execution, better assessment of the task being executed by the user can be performed (e.g., more sensor data may be gathered), strengthening the decision to transition to stairs down 502 gait activity. Once the POD 100 determines that the user is descending down stairs, it may change the way the gait dynamics is managed in view of step-over-step operation.

The POD 100 may transition from the stairs down 502 activity to the slow walk activity 501 when the thigh cyclical motion breaks pattern from what was observed during stairs down ambulation or if the thigh segment start rolling forward again. For example, the transition may occur if the angle, velocity, and/or acceleration of the thigh is above or below a threshold. In some embodiments, a direct transition from stairs down 502 gait activity to other locomotor activities may not be supported. For example, the POD 100 may have an intermediate transition to improve the mobility and comfort of the user. For example, the POD 100 may transition to slow walk 501 after stairs down 502 to account for at least a half a step or one or more steps performed after stairs down 502. Furthermore, the user may enter a downward sloping terrain after descending stairs. In some embodiments, The POD 100 may remain in the stairs down 502 activity when the user reaches the downward sloping or downward ramp terrain (e.g., the thigh segment does not roll forward past a threshold angle or position). Thus, the POD 100 may avoid unnecessary transitions, as the POD's 100 behavior would still be adequate to address the terrain.

The stairs up 503 activity may be used to manage the POD's 100 operation when the user is climbing stairs in a step-over-step fashion (e.g., one foot is placed on each stair during the user's ascent). Leveraging the POD's 100 (e.g., a powered knee prosthesis) characteristics, the stairs up 503 activity can be directly supported through the provision of net mechanical positive power to the user. The added mechanical power may make the task easier and more natural for the user. Since the user's center-of-mass may be vertically elevated as part of the functional expectations of the stairs up 503 activity, the knee may be extended under load during the stance phase, coupled with swing phase kinematics foot positioning during the stairs up 503 activity. Thus, in some embodiments, the stairs up 503 activity achieves proper man-machine synergy between the user and the POD 100 which is advantageous since the actuator controls must allow for varying balance in the respective task power contribution of the user and the POD 100.

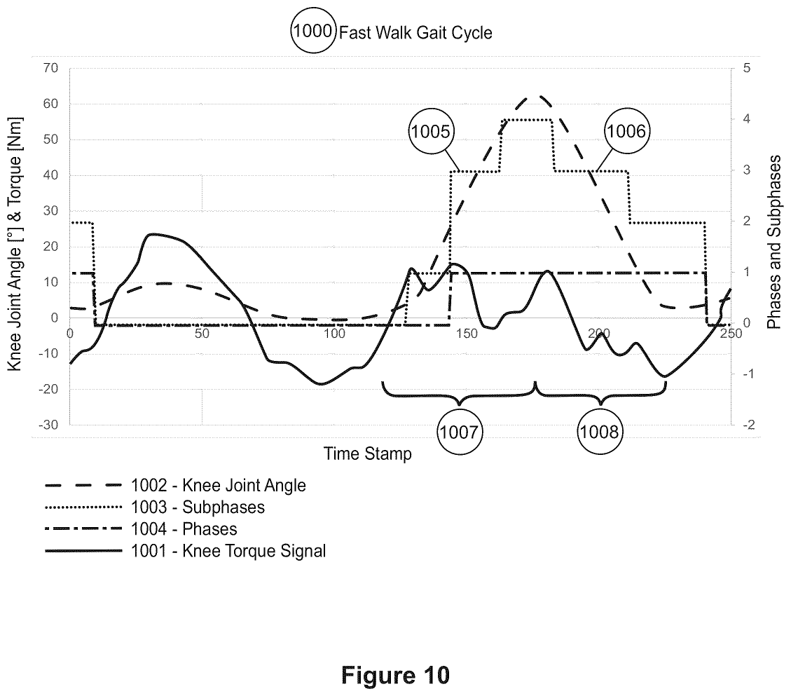

In some embodiments, the stairs up 503 gait activity may be entered from the slow walk 501 gait activity or standing static. The POD 100 may perform this transition when the user is stationary or moving slowly in a non-cyclical manner at the bottom of a staircase or uphill terrain. Similar to other transitions, the transition may be initiated when the angle, velocity, and/or acceleration of the shank and/or thigh satisfy one or more threshold. The transition may also, or alternatively, be initiated based on the measurements of one or more force sensors satisfying one or more thresholds. In some embodiments, a transition between gait activities may occur when the gait pattern (e.g., the gait cycles of FIG. 10) changes a threshold amount.

During the stairs up 503 activity, the user can voluntarily position the prosthetic foot on the first step of the staircase (bottom of the staircase) using the residual limb hip function by using the swing phase low mechanical impedance characteristics of the POD's 100 knee. Stance phase detection coupled to the POD's 100 kinematic configuration corresponding to stepping on the first step can then be used as criteria to trigger the transition to stairs up 503 gait activity. Additionally, other variables can be used to strengthen or refine the stairs up 503 transition. For example, vertical displacement between consecutive stance phase detection can be estimated using the on-board IMU and compared to a predetermined threshold. Additionally, or alternatively, measured knee torque can be compared to a predetermined threshold to verify that extension torque is being measured while in an appropriate lower-limb kinematic configuration. The stairs up 503 gait activity can also, or alternatively, be used to manage discrete obstacles like single steps and curbs, as the task functional expectations are the same and the detection mechanisms of the POD 100 can easily address the specifics of these obstacles through refinement of one or more of the thresholds used for some or many of the criteria mentioned above. Additionally, or alternatively, the stairs up 503 gait activity can be entered from the fast walk 505 gait activity. This transition may occur where the user desires to transition directly from cyclical walking gait to climbing stairs, without stopping at the bottom of the staircase. In this dynamic transition case, the user may begin climbing stairs using the sound limb, allowing detection of stairs up 503 gait activity to take place in the resulting initial swing phase cycle of the POD 100.

The stairs up 503 activity may be exited to the slow walk 501 activity when the cyclical execution of the user's gait is observed to terminate or change in a significant manner. In some embodiment, the change in the user's gait or in the pattern of the user's gait may indicate that while a cyclical gait is maintained, it no longer corresponding to the kinematics associated with the staircase terrain. In some embodiments, the stairs up 503 activity is terminated by the user's stopping gait. For example, the user may stop on his or her sound limb, allowing the control system to observe a significant delay in the cyclical gait pattern of the POD 100. The POD 100 may revert to slow walk 501 gait activity once the delay satisfies a predetermined (minimum) duration threshold. In some embodiments, the POD 100 may dynamically transition back to the slow walk 501 gait activity by reducing the (minimum) duration threshold used for the control system to identify a termination of the cyclical gait pattern. The adjusted (minimum) duration threshold may allow the user to efficiently climb staircases where sections of staircases are interrupted by landings or when a single staircase is terminated on a floor without making it mandatory for the user to pause at each terrain transition. In some embodiments, the POD 100 monitors residual limb kinematics in view of the past gait cycle events. Any change to the kinematics pattern may be compared to one or more predetermined thresholds and allow the POD 100 to evaluate in real-time the significance of the change. When the change in the kinematic pattern of the POD 100 exceeds a threshold variability associated with the terrain specifics and the user's general capacity to execute the task in a consistent and repeatable manner, the POD 100 may determine that the task has been terminated and the POD's 100 gait activity transition may be triggered. In one non-limitative example, the residual limb (maximal) hip flexion or thigh absolute angle is monitored for significant change to implement the dynamic transition out of stairs up 503 gait activity as described above.

In some embodiments, the POD 100 uses the slow walk 501 gait activity as an intermediate transition when transitioning out of the stairs up 503 gait activity. Thus, the slow walk 501 activity may be used as a buffer state to support transition and reduce or minimize reliance on over-defining all possible cases of transition between gait activities. For example, the POD 100 may be configured to default to slow walk 501 when one or more exit conditions for the stairs up 503 activity are met. The POD 100 may then transition to any other allowable gait activity as soon as the conditions for the transition to occur are detected, as described herein. In some embodiments, the POD 100 remains in the slow walk 501 gait activity for less than one gait cycle when the user is executing a dynamic transition from stairs up 503 to fast walk 505 or yield 504, for example. The POD may perform dynamic transitions using slow walk 501 as an intermediate transition when exiting other gait activities.

In some embodiments, the yield 504 activity may correspond to a general class of functional tasks for the POD 100. The various functional tasks may be grouped under a single gait activity to simplify the POD's 100 control system and make perceived task execution more consistent from the user's perspective. These general functional tasks are associated with the POD's 100 ability to flex under load, while regulating the flexion motion dynamics to predetermined amplitude level, favoring stable and safe task execution by the user. Although stability and safety are typically associated with a locked knee for users with limited residual limb hip control or power, this task management strategy may fail when the user is operating in descending terrain. For descending or uneven terrain, a failure to lower the user's center-of-mass in a smooth and controlled manner may potentially result in the user vaulting over the extended locked knee and may impair balance or prevent proper weight transfer between the residual and sound limb in a normal step-over-step gait pattern. In all these cases, a failure to properly support the functional knee task through proper management of the knee flexion under load is likely to cause the user to stumble or fall. The risk increases in the cases where the user has limited residual limb hip power or control. Thus, the yield 504 activity efficiently regulates the POD's 100 knee flexion under load to improve the mobility and safety of the user.

In some embodiments, the yield 504 gait activity may be used to manage at least (1) the stand-to-sit transition; (2) the initial stance cycle when entering a staircase downwards prosthetic limb first; and/or (3) the ramp descent stance phase cycle. The yield 504 activity may be used as a transient stance phase state to allow the POD 100 to seamlessly bridge into its final gait activity. The yield 504 activity may also, or alternatively, allow the POD 100 to gather additional user generated input. For example, the POD 100 may allow for knee-controlled flexion and conduct further testing for the task execution specifics at swing detection. The additional measurements and user input may allow the POD 100 to transition to the gait activity best suited for the terrain or task.

In some embodiments, the POD 100 may be managed through multiple gait activities as mapped by control map 500. For example, as described above, the POD 100 may use intermediate transitions when performing energy dissipative locomotion activities. It should be understood that scenario presenting similar characteristics to the scenarios described herein, such as going down a curb while standing on the prosthetic limb, going down a curb prosthetic limb first, or squatting, would be managed using the same or a similar strategy. Thus, the POD 100 may provide similar functional performance to the user without requiring a priori knowledge or programming for the specific task, which simplify device management and increase device usability.

In some embodiments, the yield 504 gait activity may be entered from 3 distinct gait activities: slow walk 501, fast walk 505 and stand up 509. In some cases, similar entry conditions are defined (e.g., threshold positions, angles, velocity, torque, etc.) for the different gait activities. The POD 100's sensor measurements may be tested against these conditions to validate whether the yield 504 gait activity should be entered. For example, based on the measured torque amplitude and/or time-based profile, combined with basic verification of the lower-limb orientation in space, the POD 100 may enter the yield 504 gait activity. A time-based profile may include how the measured torque amplitude changes over a certain time period. In some embodiments, the POD 100 may use a time-based profile to determine one or more time-based trends (e.g., a rate of change) of one or more sensor measurements. In some embodiments, comparing the POD's 100 measured knee joint torque to a predetermined threshold allows the control system to determine, through identification of (minimum) flexion torque level, that user's center-of-mass lies behind the feet and that the user is pushing into the knee to create the flexion motion. These determinations may be strong indications for the POD 100 to operate in an energy dissipative mode. However, this lower configuration and flexion torque trend may also be observed in the initial stages of level ground walking stance phase, where the knee acts as a spring, absorbing the user's weight as the load transfer over from the sound limb. In some embodiments, the time-based profile of the measured knee joint torque can be used to prevent the knee from yielding under the user's weight when the user is operating in level walking, while still making the system safe and easy to operate when the user accesses the dissipative functions.

While entering yield 504 gait activity from slow walk 501 or stand up 509 is characterized by the user's limited body dynamics and shock absorption, the situation may be different when entering from the fast walk 505 gait activity. When entering yield 504 from the fast walk 505 gait activity, the user's forward momentum may create a larger flexion torque amplitude for a short amount of time. In some embodiments, the POD 100 may use both amplitude and duration of an (minimum) amplitude threshold to determine whether to transition to the yield 504 activity. The POD 100 may also, or alternatively, use the POD's 100 configuration and spatial orientation determine whether to transition to the yield 504 gait activity.

The POD 100 may transition to the yield 504 gait activity from stand up 509 and/or slow walk 501 based on a (minimum) measured knee joint flexion torque threshold. In some embodiments, the POD 100 may transition after the torque threshold has been satisfied for a duration threshold. The duration threshold may change based on the knee torque. For example, the duration threshold may be longer (e.g., 0.25 seconds, 0.5 seconds, 1 second, 2 seconds, longer than 2 seconds, or any amount of time in-between) if the knee torque reaches a first (minimum) torque threshold, compared to the duration threshold if the knee reaches a second (minimum) torque threshold. The POD 100 may use first torque threshold when transitioning from stand up 509 and/or slow walk 501 to yield 504, and the second torque threshold when transitioning from fast walk 505. The first threshold may reflect natural and less physically taxing motions for a user operating in non-cyclical tasks or with low body momentum (e.g., while not walking fast). In some embodiments, the torque thresholds may be satisfied when the POD 100 is operating in the fast walk 505 gait activity or other locomotion task. Thus, the duration thresholds for the corresponding torque threshold may be longer than what can be observed in the slowest supported cyclical gait of the fast walk 505 gait activity or other locomotion activates to prevent inadvertent transition to yield 504. Using a higher (minimum) flexion torque threshold with a shorter duration threshold (e.g., 100 ms, 200 ms, 300 ms, 400 ms, longer than 400 ms, or any length of time in-between) allows the POD 100 to reduce or minimize possible delays associated with the yield 504 activity when dynamically transitioning from level walking cyclical locomotion to ramp descent cyclical locomotion. Thus, a short duration threshold may improve user safety, reduce or minimize constraints on the user gait style and velocity, and increase or maximize system usability.

In some embodiments, the POD 100 transitions from the yield 504 gait activity when a swing phase is detected. For example, the POD 100 may rely on another gait activity for swing phase management or transfer the POD 100 to another gait activity when the control system determines that more specific functional requirements are to be used. For example, the POD 100 may transfer out of the yield 504 activity during swing phases associated with ramp descent ambulation, either in cyclical or non-cyclical patterns. Management of cyclical or non-cyclical ambulation in ramp descent may be performed through the combination of the slow walk 501 and fast walk 505 gait activities when operating in swing phase configuration, and yield 504 gait activity when operating in stance phase and meeting the expectations introduced above for allowing the POD 100 to operate in energy dissipation mode.

Additionally, or alternatively, the selected task strategy may be evaluated on a step-by-step basis by testing for yield 504 entry conditions when in stance phase and testing for slow walk 501 or fast walk 505 entry conditions when a swing phase is detected. For example, the POD 100 may test for specific conditions based on the feedback provided by the POD's 100 knee joint sensing subsystem and may advantageously use the feedback (e.g., sensor measurements) to manage the POD's 100 transition out of yield 504. For example, the POD 100 may evaluate the sensor measurements during the prior stance phase when swing phase is detected, combined with the residual limb hip and/or thigh segment absolute orientation kinematics to identify the gait activity that may provide the best suited functional support for the user's current task and terrain. For example, in the scenario where a large amplitude flexion motion was observed during the previous stance phase and thigh segment absolute orientation has been observed to correspond to hip flexion, the POD 100 may determine that it should transition to the sit 506 gait activity. Additionally, or alternatively, if a small amplitude flexion motion was measured during the previous stance phase and thigh segment absolute orientation has been measured to correspond to hip flexion, the POD 100 may determine that it should transition to the stairs down 502 gait activity to match the task functional requirements of the user's current task and/or terrain. If small amplitude flexion motion was measured during the previous stance phase and thigh segment absolute orientation has been observed to correspond to hip extension, the POD may determine that it should transition to the slow walk 501 or fast walk 505 activity, based on the specific conditions used to determine the cyclical or non-cyclical nature of the gait task at hand. As describe above, the POD 100 may default to slow walk 501 as an intermediate activity (e.g., a transient state) to reduce and/or minimize duplicative test conditions across multiple system state or gait activities. For example, the POD 100 may transition to the slow walk 501 until the next swing or stance detection event, at which point the POD 100 may automatically test for a fast walk 505 transition or any other applicable transition.

In some embodiments, absolute orientation refers to the position of a limb or body part in relation to the surroundings of the user (e.g., rather than in relation to another limb or part of a limb). In some embodiments, the POD 100 may use one or more orientation thresholds to determine whether the residual limb hip and/or thigh segment's absolute orientation kinematics correspond to the entry conditions of one or more gait activities. The POD may determine that an orientation threshold is satisfied if the absolute angle of a limb matches the angle of the orientation threshold within a threshold degree (e.g., ±1 degree, 5 degrees, 10 degrees, more than 10 degrees, or any number of degrees in-between). The orientation threshold may correspond to a predetermined angle along one axis, two axes, or three axes. Alternatively, or in addition, the orientation threshold may correspond to the absolute angle of a limb (e.g., thigh and/or shank) or part of a limb. For example, the POD 100 may determine that an orientation threshold is satisfied if the absolute angle of a limb matches the position of the orientation threshold within a threshold amount (e.g., 10 cm, 20 cm, 30 cm, more than 30 cm, or any amount in-between). For example, the orientation threshold may be satisfied if the thigh reaches a certain height and rotation.

The fast walk 505 gait activity may define locomotion tasks where cyclical walking on a relatively flat surface is performed by the user. Under these circumstances, it is possible to improve the contribution of the powered POD 100 to the user's forward progression through active swing phase management and late stance proactive knee flexion. Thus, the POD 100 may provide large benefits such as improve speed, mobility, and efficiency to the user from the contribution of the powered knee prosthetic in cyclical level walking gait. Furthermore, the POD 100 may prevent the POD's 100 knee from being overly active which can be destabilizing for the user and reduce device usability. For example, the POD 100 may compare one or more measurements against one or more thresholds to differentiate between non-cyclical walking managed through the slow walk 501 gait activity and cyclical walking managed through fast walk 505 gait activity.

In some embodiments, the fast walk 505 gait activity can be entered through transitions from slow walk 501 gait activity or yield 504 gait activity. As described above, the fast walk 505 activity interacts directly with the yield 504 gait activity in the management of the cyclical ramp descent ambulation management, by directly handling the swing phase when the POD 100 determined that predefined conditions are met at the swing phase detection in during the yield 504 activity. Similarly, the POD 100 may transition from the slow walk 501 activity to the fast walk 505 gait activity if the POD 100 determines that a cyclical gait is taking place at a specific, or various times during the slow walk 501 activity.

In some embodiments, the fast walk 505 gait activity can be exited through five or more different transitions, which may correspond to entry condition of other gait activities described above. As shown in schematic 500, the fast walk 505 activity can be exited to the slow walk 501, stairs up 503, yield 504, and sit 506 gait activity. The transition from fast walk 505 to slow walk 501 may occur when the POD 100 determines that conditions related to maintaining a cyclical level walking gait have not been met, in which case the system reverts to slow walk 501 gait activity. The fast walk 505 activity may exit to the yield 504 gait activity when (minimum) flexion torque satisfies a torque threshold during the stance phase, as described above. The fast walk 505 and yield 504 cycles are observed when the user is operating in cyclical ramp descent, as described above. The POD 100 may transition from the fast walk 505 activity to stairs up 503 activity when the user goes directly from cyclical level walking to stairs ascent, without taking the time to stop at the bottom of the stair case. Similarly, POD 100 may transition from the fast walk 505 activity to the sit 506 activity when the POD 100 does not transition to slow walk 501 to bridge the two activities. For example, the POD 100 may not use an intermediate transition when the transitions from the fast walk 505 activity to the sit 506 activity occurred at a fast pace, which may prevent the system from transitioning to slow walk 501, or because the user did not rely on the prosthetic device to support the transition, which may be observed on a user that has sufficient physical capabilities or has developed bad use habits from the limitation of mechanical and passive knee joints. In some embodiments, if the POD 100 is overly active for the user's lifestyle or gait style, the predetermined entry and exit conditions associated with the fast walk 505 activity may be modified to make it harder to enter and easy to exit the fast walk 505 activity. The modified conditions may result in a higher correlation between the functional knee characteristic under the POD's 100 gait activity and the user's intent and may prevent possible negative consequence on the POD's 100 usability.

The stand-up 509 activity corresponds to the general activity during which the user transition from the seated position (e.g., the sit 506 activity) to the standing position. In some embodiments, the standing position may be managed by the slow walk 501 gait activity (e.g., the POD 100 transitions to the slow walk 501 gait activity after the stand-up activity is complete). The stand-up 509 activity may use active management (e.g., provide positive mechanical power) and a specific gait activity designed to support the standing up action to support the user and provide a more efficient transition. Similarly to what can be observed while the task is being performed by non-amputee, the role of the POD's 100 knee during the stand-up 509 activity is to actively extend under load, allowing the user's center of mass to rise to its full elevation associated with standing.

In some embodiments, the stand-up 509 activity may be entered from the sit 506 activity through the sit-to-stand transition 510. Because of the functional requirements of the POD 100 during the sit-to-stand transition 510, the stand-up 509 activity may include a stance phase with no swing phase. In some embodiments, a swing phase may not be supported as the POD 100 may not need to meet any swing phase functional requirements during the stand-up 509 activity. Several mechanisms can be used to detect the sit-to-stand transition 510 while operating in the sit 506 activity.

In some cases, detection of the stance phase may be a prerequisite for the sit-to-stand transition 510 to occur. Additional conditions (e.g., measurements thresholds or ranges) related to a general change of body posture and/or extension of the POD's 100 knee joint may be used to detect a stance phase more accurately, without causing excessive limitation on the flexibility of the detection mechanism to adapt to the variability in users' movement and transitioning preferences, physical capacity, or other physical limitations.

In some embodiments, the POD 100 initiates the sit-to-stand transition 510 (from the sit 506 activity) when it detects (1) a stance phase (e.g., contact between a user's lower limb and the ground) and (2) an extension of the hip joint (on the leg that includes the POD 100). As described herein, in certain cases, the POD 100 can determine the position and/or movement of the user's thigh (on the leg that includes the POD 100) by comparing measurements from the knee joint (e.g., knee angle, knee velocity, knee torque) and measurements from an IMU positioned on the second limb (e.g., position relative to ground, absolute angle, rotation relative to ground, rotational velocity relative to ground, forward velocity, etc.).

In some cases, the POD 100 may enter the sit-to-stand transition 510 if it detects a load on the POD that satisfies a load threshold and detects hip extension. In some cases, the load threshold may be zero kg, 1 kg, 5 kg, 10 kg, 20 kg, more than 20 kg, etc. As described herein, in some cases, the POD 100 may detect hip extension by comparing the absolute angle of the shank (e.g., second limb member) with the relative angle of the knee joint. For example, if the absolute angle of the shank remains unchanged (or varies by less than a threshold amount, such as, but not limited to 1-2 degrees) while the relative angle of the knee decreases beyond a threshold amount (such as, but not limited to five degrees) indicating knee extension, the POD 100 may determine that the hip is extending. In certain cases, using the absolute shank angle and relative knee angle enables the POD 100 to quickly and accurately detect that a user is beginning to stand up from a sitting position.

In certain cases, the POD 100 may enter the sit-to-stand transition 510 after the user has already begun standing up. For example, if a user begins standing up with his or her non-prosthetic limb (e.g., the user does not initially use their prosthetic limb for support), the POD 100 may delay the sit-to-stand transition until the POD 100 detects a force that satisfies a load threshold (e.g., the POD 100 detects that the user is placing weight on the POD 100). In this scenario, the POD 100 may detect hip extension (e.g., using absolute shank angle and relative knee angle) before detecting stance phase. By allowing the entry conditions to be met in various orders, the POD 100 may dynamically provide active standing-up assistance without reducing a user's movement and/or flexibility.

In certain cases, the POD 100 may detect the stance phase detection and hip extension detection asynchronously. For example, after detecting stance phase, the POD 100 may initiate a detection window. The detection window may have a duration of one second, two seconds, three seconds, five seconds, ten seconds, more than ten seconds, or any amount of time in between. In some cases, the detection window may continue even regardless of any change on the load of the POD 100. During the detection window, the POD 100 may enter the sit-to-stand 510 transition if it determines that one or more entry conditions have been met (e.g., detects hip extension, knee extension, and/or a change in knee joint angle that satisfies a knee angle threshold).

In some embodiments, the POD 100 may initiate a detection window if (1) a stance phase is detected, (2) the shank (or second limb member) satisfies an absolute angle threshold or position threshold, and/or (3) the knee angle satisfies a relative knee angle threshold.

In certain cases, the POD 100 may use one or more angle and position thresholds to confirm that the position of the second limb satisfies a position threshold, or the shank satisfies absolute angle threshold. For example, if the absolute angle of the shank is less than 5°, 10°, 15°, or 20°, etc., the POD 100 may determine that the shank satisfies the absolute angle threshold and/or the shank position threshold.

In certain cases, the POD 100 may determine that the knee angle satisfies a relative knee angle threshold if it determines that the knee angle has decreased by at least five degrees within a time period (e.g., one second).

In some embodiments, the position threshold may be satisfied if the distance between the center of mass of the residual limb and the center of mass of the second limb is less than a maximum distance threshold. The maximum distance threshold may vary according to the physical characteristics of the user (e.g., height, leg length, etc.). Once one or more conditions are met, the POD 100 may initiate a detection window as described above.

In some cases, the POD 100 may not use a detection window. For example, the POD 100 may cancel the sit-to-stand transfer 510 if a stance phase is no longer detected.

In some embodiments, the POD 100 may have two transition paths to exit the standing-up 509 activity. In the first path 512, the sit-to-stand 510 transition finishes so that the user is standing with the POD's 100 knee in a sufficiently extended position, so that the user may transfer his or her weight to the ground in a relatively equal manner between the prosthetic and non-amputated limb. Under the first path 512, the conditions for the POD 100 to exit the standing-up 509 activity may be met when the knee extension satisfies (e.g., exceeds) an extension threshold. In some embodiments, when the extension threshold is met the POD 100 may enter the slow walk 501 activity. It should be understood that standing still may be a subset of the limited ambulation gait activities covered by the slow walk 501 activity.

In the second transition path 513, the POD 100 determines that the standing-up 509 activity execution is stopped before completion and the user has reverted to sitting-down. This transition path 513 may occur if the standing-up 509 activity is interrupted or the external environment does not allow the standing-up 509 activity to be execute in an unconstrained manner, for example when the user is sitting close to a fix table or when using walking aids. Under the transition path 513, the POD's 100 knee joint is observed to move towards flexion, which triggers a transition to the sit 506 activity. The POD 100 may first transition to the yield 504 activity as an intermediate transition before transitioning to the sit 506 activity.

As described above, the POD 100 may provide the benefit of being able to restore loss muscle function of the lost joint. In one embodiment, a POD 100 is used to replace the lost function of the knee joint during the sit-to-stand activity. Similar to most locomotor tasks, non-locomotor tasks like sit-to-stand are characterized by a variability that individuals without amputations typically manage instinctively and without conscious thought. To replicate the ability of the human neuromotor control system to deal with varying tasks and environments, specific attention must be directed to both the user-to-user variability in task execution, as well as the environmental constraints and their effect on the task implementation. For a prosthetic device to properly support this type of task, the control system must inherently account for this level of variability. Thus, in some embodiments, the POD 100 may use an adaptive sit-to-stand control scheme.

Some embodiments of the POD 100 disclosed herein are directed towards a powered POD 100 with a control system subphase that supports the portion of the sit-to-stand activity during which the knee joint of the POD 100 is extending. For a powered POD 100, extension may correspond to a period during which the powered POD 100 provides positive mechanical power to the user. In some embodiments, the control system may use activities and/or subphases (e.g., different modes and submodes) to adapt to the frequent interruptions and variability that are expected to occur during a knee extension cycle. Thus, the POD 100 may use the different gait activities and subphases to deliver power in a more constant, flexible, and efficient manner to the user. Additionally, the gait activities and subphases of the control mechanism allow the POD 100 to be reactive to slight changes in the user behavior, instead of being point-to-point programed. Thus, the control system prevents the POD 100 from having rigid behavior that may reduce user comfort, mobility, and safety.

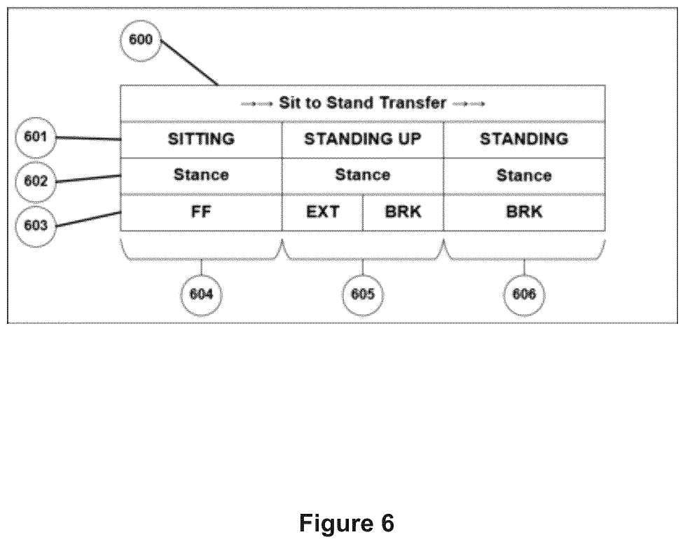

FIG. 6 presents a diagram illustrating certain phases and subphases associated with the sit-to-stand transition 510 and neighboring activities. As described above, the sit-to-stand transition 510 may manage the POD's 100 behavior while the user is going from the seated position to the standing still position. The schematic 600 of the activities 601, phase 602, and subphases 603 illustrates an example of how the control system may sequentially flow through the specific activities of sitting 604, standing-up 605, and standing 606.

While the user is seated, the POD 100 operates under the sitting activity 604, where the POD 100 may be in either stance or swing phase. During the sitting activity 604, a functional expectation for the POD's 100 knee actuator may be to be compliant and allow the user to sit comfortably without being restricted in his or her movement by the POD 100. Thus, during the sitting activity 604, the subphase may be maintained as FF, which allows the expected level of compliance using torque feedback to produce a low apparent impedance.

In some embodiments, the POD's 100 control system has a standing-up subphase 605 to support the portion of the sit-to-stand transfer during which the knee joint is extending. In some embodiments, the sit-to-stand transfer corresponds to the period during which the POD 100 provides positive mechanical power to the user. During the standing-up activity 605, the POD 100 may adapt the power delivery to frequent and/or unexpected interruptions of the standing-up task (e.g., the user is sitting at a fixed table and is to slide out the POD 100 from under the table). Additionally, the POD 100 may respond to slight changes in the user's behavior and adapt power delivery to improve the mobility, efficiency, and safety of the user.

While operating in the sitting activity 604, a user's weight transfer to the prosthetic limb may cause the POD 100 to transition to stance phase, which may cause the POD 100 to transition to the standing-up 605 activity upon detection of knee extension motion combined with residual limb thigh segment forward tilt motion (e.g., thigh segment becoming more vertical). Since the POD's 100 is providing low mechanical impedance at the actuator level, the user can move the knee under load with little effort, which allows for an effective detection mechanism. In some embodiments, the POD 100 may transition to the standing-up 605 activity based on the conditions listed above and with no respect to the observed phase (e.g., swing or stance). The swing phase provides greater flexibility and balance to the user, especially when the POD's 100 mechanical configuration has the knee highly flexed and the user's center of mass far behind the user's feet. Thus, the transition to the standing-up 605 activity may occur in either stance or swing phase.

As shown in FIG. 6, two distinct subphases (e.g., EXT and BRK) may be used to manage the stance phase of the standing-up 605 activity. In some embodiments, the standing-up 605 activity may not present a typical swing phase, so the POD's 100 activity during the standing-up 605 activity may be covered by these EXT and BRK subphases. In some embodiments, detection of a swing phase transition would be addressed by the activity management flowchart of FIG. 5 and result in a direct transition to slow walk (FA) 501. In some embodiments, entering the standing-up 605 activity may cause the POD 100 to default the subphase to EXT (extension) (from FF). In some embodiments, the POD 100 uses proportional control during the EXT subphase. For the proportional control, the POD 100 may use a relative knee angle of zero (e.g., the first and second limb members are colinear) as the target angle. The EXT subphase may be maintained until the BRK (braking) subphase is triggered.

In some cases, the BRK subphase may be used to decelerate the rotation of the POD 100 and to smoothly transition to the standing activity 606. The POD 100 may enter the BRK subphase when the POD 100 determines that the relative knee joint angle satisfies (e.g., is less than) an angle threshold during knee extension. The angle threshold may be 25 degrees, 20 degrees, 18 degrees, 10 degrees, 5 degrees, less than 5 degrees, or any angle in between.

Similar to the TOA subphase described herein, the BRK subphase may use velocity control to decelerate the rotational extension velocity of the knee joint quickly and smoothly. The POD 100 may use a target velocity of zero or near zero for the velocity control. Furthermore, the velocity control may behave as a damper and prevent the POD 100 from hitting a mechanical barrier (e.g., the POD's 100 kneecap). In some embodiments, the BRK subphase may be similar to or use one or more of the features of the TOA subphase. The TOA subphase will be discussed in more detail in a further section. In some embodiments, the BRK subphase may be maintained during the standing activity 606 after transitioning from the standing up 605 activity.

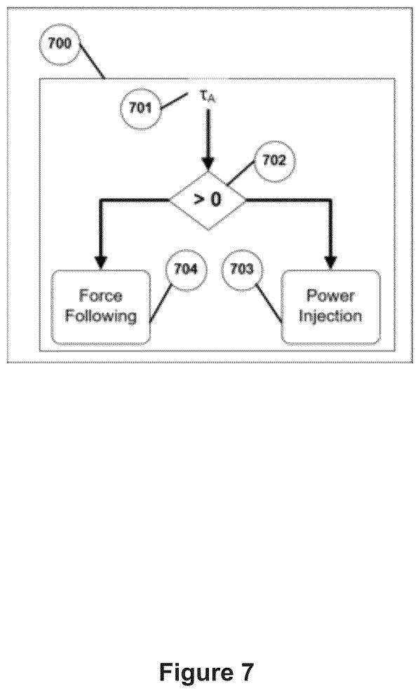

FIG. 7 presents an example of an embodiment of a schematic of an EXT subphase actuator behavior management process 700. The EXT subphase is used to manage the POD's 100 operation when the prosthetic knee is extending. To properly manage the extension motion without creating artificial constraints that could lead to reduced usability or excessive balance or hip control expectations from the user, a powered prosthetic may implement flexible actuator behavior during the EXT subphase. On one hand, the POD's 100 actuator may actively support the role of the POD's 100 knee in the overall body dynamics and balance, while on the other hand, avoid imposing hard constraints on the way the sit-to-stand task is executed by the user. The combination of the control system and fundamental actuator behavior may be sufficiently reactive and flexible to account for the large number of variables affecting the exact power delivery pattern, timing and execution of the sit-to-stand task.

Thus, the EXT subphase may address two main operational scenarios in which the functional constraints and the contribution of the user may be widely different. On one hand, the user may rely heavily on the POD 100 for support and proper lower-limb kinematics. In this first scenario, the knee is expected by the user to provide positive mechanical power and actively support the sit-to-stand task execution, with certain (minimal) inputs being provided by the user. On the other hand, it is possible that the user is more skilled, shows stronger residual limb hip power, or is generally in a situation where it is possible to perform the sit-to-stand task with reduced and/or minimal positive mechanical power from the knee prosthesis. In this second scenario, the POD 100 may adapt its behavior such that it does not limit the user's capacity to execute the transition under full control.

It is to be noted that while it is possible that the exact scenario under which the sit-to-stand task takes place is dependent on the user activity level or physical capacity, it may not be the sole variable affecting the task execution. Environmental variables such as physical characteristics in which the sit-to-stand task is being executed may also play a role in the strategy adopted or forced onto the user. It is common for both amputees and non-amputees to change the pace at which the task is being executed or their frontal plane balance (e.g., due to presence of obstacle). In some embodiments, the EXT subphase is dedicated to managing the sit-to-stand transfer prosthetic knee task associated with knee extension. In the case where the user is to stop the sit-to-stand task and revert to the stand-to-sit task, this may cause an activity transition detection and may be managed as illustrated by FIG. 5.

The variability in the operational scenarios during the sit-to-stand activity may result in actuator behavior expectations, which can be represented as discrete operational states, as shown in FIG. 7. In some embodiments, the EXT subphase actuator behavior management process 700 may be the decisional process used by the POD 100 during the EXT subphase. The EXT actuator behavior management process 700 may control the POD 100 during the EXT subphase so that it efficiently implements the functional expectations associated with the standing-up activity 605, while improving usage of the POD's 100 capacity to provide positive mechanical power, without affecting the user's capacity to impose specific kinematics to the POD 100. Thus, the EXT actuator behavior management process 700 may effectively address the functional expectations of the EXT subphase and implement adequate actuator behavior. In some embodiments, the process 700 uses torque feedback measurement to determine which operational scenario is currently taking place and determines whether the POD 100 should operate in force following 704 or in power injection 703 mode. The force following 704 mode and power injection 703 mode will be described in more detail below.

In some embodiments, the EXT actuator behavior management process 700 may control the nature of the power exchange between the POD 100. For example, the process 700 may modify the power exchange based on the measured knee torque value 701 that corresponds to the POD's 100 joint. In some embodiments, and as described in more detail below, the process 700 may manage the POD's behavior based on a single decision or comparison. For example, the process 700 may compare the measured knee torque value against a null torque 702 to determine whether the prosthetic knee is acting as kinematics generator or whether the user is. In the case where the measured knee torque value 701 is positive, the process 700 may implement power injection 703 actuator behavior. In some embodiments, positive or flexion torque value may indicate that the user's weight is resting against the knee joint actuator extension motion and the user is using the knee for support. In the case where the measured knee torque value 701 (e.g., the interaction torque) is measured to be smaller than 0, this may indicate a general interaction torque in the extension direction. For example, the POD's 100 knee may be moving under the drive of the user's residual limb which may cause the POD's 100 knee to extend faster than it would under the torque of the motor 205 alone. In some embodiments, the negative measured knee torque value 701 indicates that the user is defining the kinematic response of the POD 100 and that the actuator is to implement the force following 704 behavior to reduce or minimize the impairment of the user motion and activity completion. Thus, where the measured knee torque value 701 is negative, the process 700 may use force following 704 mode.

Alternative decision schemes or measurable/estimable quantities could be used to support the EXT actuator behavior decision process. While comparing the threshold (e.g., the null torque value 702) to a fixed value provides a simple correlation to the physics at hands and the user's task, more advanced decision-making mechanisms may prove better suited in specific implementation cases. For example, the measured knee torque value 701 may be compared against one or more hysteretic thresholds. The hysteretic thresholds may have the added capacity of using selective mechanisms when moving from one actuator behavior to the other, or implement non-symmetrical transitions, for example. The process 700 may also use variable threshold values so that the POD's 100 overall behavior may be based on an external variable (e.g., knee flexion angle, absolute thigh angle, absolute shank angle, vertical ground reaction force, etc.). Hence, in some embodiments the POD biases the amount of extension torque to cause the system to implement the force following behavior 704 for higher knee flexion angle, such that the control system response favors support for the lower limb mechanical configurations that are harder on the user residual limb.

Additionally, non-null threshold values could also be used, for example, in the case where a fixed actuator behavior bias would be found clinically effective in managing the EXT subphase. Use of a positive non-null threshold value may result in higher flexion torque for the system to adopt the power injection 703 behavior, leaving the POD's 100 knee joint actuator operating in force following behavior 704 for low flexion torque values and relying more on the user for the joint task management. Use of a non-null negative threshold value on the other hand may result in more user input and larger extension torque for the POD 100 to transition to the force following actuator behavior 704, which could be indicated for clinical cases where limited residual limb hip power is observed to be available.

In some embodiments, the POD 100 uses a learning mechanism as part of the multi-layered controller's 300 learning layer 110 to define the threshold value, or threshold regression to a specific device variable, based on data collected from execution of the sit-to-stand transition 510 through clinical or day-to-day usage of the POD 100. For example, the control system may use a cost function to define the desirable behavior or performance metric to modify the threshold value based on the recorded gait activity occurrences. In some embodiments, the threshold value may be modified in real-time (e.g., while the user is using the POD 100). In some embodiments, a cost function assigning a performance penalty when the extension torque exceeds a predefined range or threshold (maximum) value could be defined and used to lower the torque threshold point used in EXT actuator behavior management process 700. For example, measuring large extension torque during EXT subphase execution may be symptomatic of a control system that is biased towards the power injection actuator 703 behavior, impairing the user's capacity to specify the POD's 100 knee joint kinematics for (optimal) task execution. Thus, the POD 100 may use the cost function to lower the threshold value used by process 700 so that the POD 100 transitions to force following actuator behavior 704 under a wider range of threshold values, providing more direct controls to the user and making the task execution more efficient.

Alternatively, or in addition, to measured knee torque value 701 (e.g., the actuator measured torque), other measured or estimated variables can be used to manage the EXT actuator behavior. While the knee's actuator torque value provides the benefit of directly quantifying and qualifying the interaction between the user and the POD 100, other sensor streams may be used in a similar fashion. For example, if the POD's 100 knee joint stiffness or mechanical impedance is known, the knee joint angle or knee velocity could be used to similar purpose (e.g., as a threshold value). Inertial measurements units output such as the absolute angles or rotational velocities of the thigh or shank segments may also, or alternatively, be used to compare the relative velocity or angle of the segments adjacent to the joint and determine whether the user is driving the joint kinematics or the actuator is driving the joint kinematics.

Based on the specific measurable or estimable quantity used to manage the EXT actuator behavior and/or the sensor characteristic supporting the process, some signal conditioning may be performed before testing the measurable or estimable quantity as part of the management process 700. Due to the large difference in the EXT actuator behavior associated with the sit-to-stand 510 activity, rapid transitions between the two actuator behaviors caused by a noisy signal could negatively affect the POD's 100 performance, generating instability and possibly preventing the user from completing the gait activity as intended. To prevent such transitions from occurring, signal conditioning such as low pass filtering may be beneficial used on the overall control scheme performance. For example, the control system may have one or more digital or analog filters.

In similar fashion, it may be preferable to add a dead band around the threshold point value used in process 700. In the case where rapid transitions of the measurable or estimable quantity (e.g., the measured knee torque 701) are observed to be caused by the user's behavior and/or input to the control system, the POD may use a dead band to establish a threshold (or minimum) difference between the threshold value and the measurable or estimable quantity for the actuator behavior to be modified. This implementation may be similar to the use of hysteretic thresholding as a decision mechanism.

Additionally, in some embodiments, it may be found beneficial from a clinical performance perspective to transition the actuator behavior from force following 704 to power injection 703, and conversely, over a finite period of time. Again, due to the significant difference in the actuator behavior to support the EXT subphase in sit-to-stand gait activity, direct and immediate transition between the two could surprise the user, generating instability and possibly preventing the user from completing the gait activity as intended. Using a finite, but small, period of time to transition the impedance controller gain through a fixed gain schedule allows the POD 100 to mitigate and smooth out the actuator behavior change as perceived by the user, without compromising the clinical function and performance. In some embodiments, the POD 100 may use cross-fading between the two behaviors. For example, the POD 100 may cross-fade the force following 704 and power injection 703 behaviors over a period of 250 ms is smooth-out the actuator behavior change, while providing satisfactory functional performance.

It is to be noted that while FIG. 7 presents the POD's 100 behaviors as separate discrete states, in some embodiments, similar outcomes may be obtained by creating a mechanical impedance map not relying directly on finite impedance states. In such an impedance map, an input variable indicative of the interaction between the user and the POD 100 (e.g., torque feedback) may be used to determine the actuator mechanical impedance based on a predefined range of impedance. In some embodiments, the direct coupling may be used to gradually vary the POD's behavior. Additionally, or alternatively, a number of intermediate states could be created the correspond to predefined intermediate impedance values based on the discretization of the amplitude of the interaction variable.

In one non-limitative embodiment, the torque signal measured between the actuator output and shank segment of the POD 100 may be used to quantify the nature and amplitude of the interaction between the user and the POD 100. Based on the amplitude of the measured torque, or its comparison to one or more predetermined thresholds, the POD 100 directly determines which actuator behavior is best suited to support the task execution by the user.

For example, in the scenario where the user is observed to rely heavily on the POD 100 for support and lower-limb kinematics, the POD's 100 sensors may measure the interaction torque as flexion torque (positive value). Since part of the user weight is resting on the POD 100, which in turn is deploying positive mechanical power to create the relevant (or required) extension kinematics, the sensor may measure flexion torque. The POD 100 may maintain the power injection 703 actuator behavior due to the measure flexion torque.

In the operational scenario where the user is observed to be able to drive the task kinematics through residual limb hip extension power, the interaction torque may be measured as extension torque (negative value). In this scenario, as described above, the user residual limb may move faster than the POD 100, which may cause the POD's 100 knee actuator to be back driven. Under such circumstances, the actuator may behave as the load and the user's residual limb may provide the mechanical power source. In some embodiments, the POD 100 may reduce or minimize the positive power expectation from the user by immediately switching the POD's 100 behavior to force following 704 upon detection of significant negative interaction torque.

As described above, continuous monitoring of the interaction torque when operating in EXT subphase allows the POD 100 to dynamically switch between the force following 704 and power injection 703 actuator behaviors. This allows the POD 100 to directly adapt to the user's task strategy and/or change of strategy during the standing-up activity 605. The POD 100 may use the decisional process (e.g., process 700) described above to determine when to modify the control scheme of the POD 100. In some embodiments, the POD 100 may modify impedance controller gains to modify the force following 704 behavior and/or the power injection behavior 703.

The force following 704 behavior may correspond to the subphase of the same name (see FIG. 6). As described above, the force following 704 behavior may also, or alternatively, correspond to the EXT subphase. In the force following 704 mode, the resulting mechanical impedance of the POD 100 may be null or low enough such that the actuator output can be easily manipulated by the user (driven or back driven) using low force. By setting the impedance controller gains properly, it is possible to use the torque feedback to indicate how much the motor 205 should move to follow the external perturbation force, making the inherent characteristics of the actuator system (friction, back driving torque, inertia) transparent. In the context of the POD's 100 operation, this means that the POD 100 can be easily back driven by the user residual limb motion.

The power injection 703 behavior is where positive mechanical power is generated through a finite impedance level, which is configured through proper definition of the impedance controller gains. While operating under power injection 703 behavior, the POD 100 can move under load and support functional tasks, such as what is observed during sit-to-stand activity. With appropriate scaling of the impedance controller gains, varying degrees of power injection and/or resulting kinematics can be obtained, in view of the finite nature of the perturbation torque/loads to be expected for given task scenarios.

Various impedance controller gains configurations can be used to achieve the desired POD 100 kinematics output, either as feedforward or feedback control schemes. For example, the POD 100 may use an open-loop torque command to generate the extension motion, which may either be fixed in time or with respect to the POD's 100 knee position. Additionally, or alternatively, a closed-loop velocity control scheme may be implemented using the general impedance controller scheme presented in FIG. 4 by setting the position gain to 0 and the mass gain to unity (e.g., 1).

To properly support the POD 100 activities, a control scheme may be selected that provides the proper kinematics, but that can also be adapted through simple adjustments to account for user variability and personal preferences. Additional considerations may be directed towards avoiding a control scheme that would create artificial constraints and that may require the control scheme be accurately mapped to the user's varying expectations or presents specific requirements on how the task is executed. For example, the proper knee joint kinematics could be generated using a position control scheme. This may, however, result in defining endpoints between which the motion takes place. The user may then be required to follow the defined endpoints when executing the task. Since the nature and variability of the sit-to-stand task does not allow the POD 100 to define repeatable end-points in knee joint coordinates, use of different control paradigms, as describe herein, are more advantageous (e.g., provide more flexibility, safety, and mobility to the user).

The process 700 allows the POD 100 to use a motion control scheme that controls the positive power injection, without setting hard boundaries. Additionally, combining the motion control scheme with the dual motion control behavior implemented by the EXT subphase allows the POD to achieve a high-level control paradigm (e.g., the control system determines and manages the user's intent, while providing enough transparent low-level function such that the user does not have to spend a lot of cognitive energy to control the POD 100). While the open-loop torque command approach directly defines the level of power injection implemented by the POD 100, this approach presents a level of abstraction that is harder to grasp and connect to the user's feedback or knee joint behavior expectation. On the other hand, similar outcomes are obtained using a closed-loop velocity controller, which also presents the benefit of a strong physical correlation, allowing the user to be able to clearly express performance expectations. Being able to represent the performance expectations in simple, physical terms bring additional value since it greatly simplifies the initial system configuration where the targeted velocity amplitude for the task execution may be defined through a trial-and-error adjustment routine.

To achieve a robust and smooth actuator behavior while the impedance controller gains are being changed to enforce the desired subphase and corresponding actuator behavior based on the decision process shown in FIG. 7, specific attention may be directed at how the controller gains are dynamically modified. Direct transition of the controller gains between the values associated with the force following 704 and the power injection 703 actuator behavior may cause discontinuities in the POD's 100 output and behavior, which may destabilize the user and potentially cause a fall.

Managed transition between the force following and power injection actuator behaviors can be achieved through scheduling of the gains transition, such that the gains are gradually moved from their current value to the targeted value associated with the other behavior, without creating discontinuity in the actuator behavior. For this strategy to be effective and not leave the actuator in transitioning behavior up to the point where it would be noticeable by the user, transitions duration in the general order of 100 ms are considered adequate. In some embodiments, the transition duration may be shorter than 100 ms. For example, the transition duration may be 20 ms, 30, ms, 50, ms, or 90 ms. Similarly, the transition duration may be longer than 100 ms.

The BRK subphase is the second subphase used to manage stance phase during the standing up 606 activity. While the EXT subphase may be directed at extending the knee to support the functional and kinematics requirements of the standing up 606 activity, the BRK subphase may be used to manage the transition to the standing activity 606, in which the knee is to stabilize at a mostly fully extended position, while gradually reducing extension velocity and stopping at the proper flexion angle, without hitting the extension stop. The role of the BRK subphase may be to prepare the activity transition toward the standing 606 activity. Due to the two actuator behaviors implemented during the EXT subphase, the varying contribution of the user to the extension dynamics through the residual limb, and the reducing knee joint loading as the knee extends, the BRK subphase may be used to prepare the activity transition (e.g., to standing 606 and/or stand up 509).

As describe above, the FIG. 6 is a schematic 600 of the sit-to-stand transfer. In some embodiments, when the user is seated, loading the POD 100 may cause a change of reading in the ground contact sensor. The change of reading may trigger the POD 100 to enter the stance phase. In the sitting activity 604, stance phase detection causes the POD 100 to enter the force following subphase, which implements the force following POD 100 behavior. When the POD 100 is in FF mode, the POD's 100 knee is mainly free to move under the control of the user's residual limb. Thus, during FF mode, it is possible for the user to initiate the body motion associated with standing-up from the seat. Namely, the user would move his or her center of mass forward, stiffen the hip joints and slowly start to extend the knees.

Measurement of the general change in posture, combined with the extension of the prosthetic knee joint under the control of the user's residual limb may trigger the POD 100 to transition from the sitting activity 604 to the standing-up 605 activity. As described herein, the POD 100 may determine the posture of the user (or thigh position/movement) by comparing measurements collected at the knee joint (e.g., relative knee angle, etc.) and measurements of an IMU or other sensors positioned on the second limb (e.g., absolute angle).

In some embodiments, with the prosthetic limb still loaded by the user, the POD 100 maintains the stance phase operation and enters the EXT subphase. The POD may use power injection 703 actuator behavior as a default when entering the EXT subphase, forcing the knee to extend under a fixed velocity closed-loop control scheme. For example, the POD 100 may adjust the actuator according to a velocity set point until a knee angle threshold is satisfied. In some cases, the POD 100 may adjust the power provided to the prosthetic knee based on feedback received from a velocity sensor at the knee joint and/or based on the rate of change of a measured knee angle. The feedback may enable the POD 100 to maintain a desired extension velocity (e.g., rotational velocity).

It is to be noted that other motion control schemes can be implemented to implement the power injection 703 behavior. For example, a torque feedforward scheme may be used instead of the closed-loop velocity. Similarly, the POD 100 may use a position signal, or other measurement, as the feedforward signal for the torque feedforward control scheme. In some embodiments, the torque feedforward control scheme may use multiple feedforward signals (e.g., position, velocity, etc.).

Fixed gain position control using a targeted position set-point close to full extension may also, or alternatively, be used and may generate adequate performance. Thus, the POD 100 may use a motion control scheme that provides POD 100 extension kinematics in a pattern coherent with the role played by the POD's 100 joint during sit-to stand activity 510. Using the power injection 703 actuator behavior as a default may allow the POD 100 to provide support to the user in the initial stages of the hip and knee joint extension, where the extension velocity is still low and gaining momentum may prevent the user from losing balance. Additionally, defaulting to power injection 703 actuator behavior provides an implicit test of the operational scenario currently adopted by the user, as the POD's 100 extension torque may extend until it meets the load imposed by the user's body weight and the resulting change in the torque measurement may be used by the POD 100 as a positive indication of the scenario at hand.

After transitioning to EXT subphase, the POD 100 may test the measured knee torque 701 (through the decision block of process 700) to decide whether to remain in power injection 703 actuator behavior or to switch to the force following 704 actuator behavior. As described herein, if the measured torque indicates flexion, this indicates that the knee is supporting the user's load and the POD 100 should maintain the power injection 703 actuator behavior to support the POD's 100 continued extension. In the early stages of the standing-up activity, when the user's body weight and/or center of mass is at the threshold (maximum) distance from the knee joint center or supports and the knee joint is at its threshold (maximum) flexion angle for the task, the POD's 100 may maintain the power injection actuator behavior until sufficient knee extension is measured (e.g., about eighteen degrees).

In some embodiments, when the POD 100 determines that measured torque is transitioning to extension torque (e.g., the torque switches from a positive to a negative value) while operating in the EXT subphase, the POD 100 may dynamically switch to the force following 704 behavior. As described herein, the transition to extension torque (e.g., a negative torque) may result from the user's center of mass getting closer to the knee joint axis, the knee having extended to a level where the user's mechanical leverage allows him or her to better use the residual limb hip power, or the user having sufficient residual limb hip power to perform a fast standing-up knee extension cycle without much assistance from the POD 100.

In some embodiments, the POD 100 may determine that the user is moving (e.g., standing up) faster than the POD 100 is extending when it detects the extension torque. For example, the force exerted on the knee joint due to the user standing up may exceed the force on the knee joint by the actuator. As such, the POD 100 may determine that the POD's 100 joint is resisting the knee extension motion imposed by the user's residual limb (e.g., when the measured torque meets a threshold that indicates that the measured torque is extension torque). In some such cases, the POD 100 may switch the actuator behavior to force following 704 to reduce or minimize the resistance imposed by actuator, thereby allowing the user to complete the standing-up task with reduced interference from the actuator. Thus, the POD 100 may enable the knee joint actuator to manually rotate faster than the POD's 100 rotational extension velocity set-point.

In some cases, if the rotational extension velocity of the knee joint is above a threshold extension velocity, the POD 100 may switch the actuator to the FF 704 actuator behavior. For example, if a use is standing up quickly, the POD 100 may detect that the knee angle is changing faster than expected (e.g., as per the velocity set-point). In some such cases, the POD 100 may alter the behavior of the knee joint actuator so that it is in an FF 704 behavior during at least the EXT subphase. In this way, the knee joint can become more compliant and move at the speed of the user rather than enforcing a preset speed.

While in the FF 704 behavior during the sit-to-stand transition, the POD 100 may determine that additional support would be helpful to a user. For example, a user may lose balance or begin to fall back while standing up and while the actuator is in an FF 704 behavior. In some such cases, the POD 100 can switch the actuator to be in a force injection mode and inject power into the knee joint to provide support and/or to help the use complete the sit-to-stand transition.

In some such cases, if the POD 100 switches the actuator to the FF 704 actuator behavior, it may continue to monitor the velocity and/or torque at the knee joint. In certain cases, if the POD 100 determines that the knee joint extension velocity (while in FF 704 behavior) decreases and/or decreases more than a threshold amount, the POD 100 may switch the actuator to a force injection behavior and inject power into the knee joint (e.g., using an extension velocity and/or position set-point as described herein). In this way, POD 100 can provide support to a user.

As another example, if while in the FF 704 actuator behavior during the sit-to-stand transition, the POD 100 detects an external flexion torque (e.g., a downward force or increased downward force on the knee joint), the POD 100 may switch the actuator to a force injection behavior and inject power into the knee joint (e.g., using an extension velocity and/or position set-point as described herein). In this way, POD 100 can provide support to a user.

Once the knee has reached a position close to full extension (e.g., a relative knee angle of less than eighteen degrees or an absolute angle of the second limb of less than eighteen degrees), the subphase management system (e.g., the control system of the POD 100) may automatically make the POD 100 transition to the BRK subphase. In some embodiments, the POD 100 may transition to the BRK subphase at different angle thresholds (such as but not limited to ten degrees, fifteen degrees, twenty degrees, etc.) to accommodate different users' needs and preferences. As described above, the BRK subphase may implement POD behavior that counteracts the momentum gained by the user while completing the hip and knee extension motions. Failure to properly counteract the momentum gained by the user may result in terminal extension impact, or overshooting of the desired knee extension position, and may fail to generate a comfortable and stable standing configuration. In some embodiments, the POD 100 configures the impedance controller of FIG. 4 to implement both velocity and force controls in parallel during the BRK subphase. The parallel impedance control terms may allow the POD 100 to address the case where the extension terminates with significant velocity, which is addressed by the velocity control with targeted null velocity, or the case where the extension terminates with low velocity, which then relies on the torque control term to control the deceleration since the velocity control term contribution may be reduced or minimal when the magnitude of the velocity is low. Thus, the POD 100 uses the BRK subphase to provide a safer and more comfortable experience for the user.

When the POD 100 reaches an almost fully extended position (e.g., knee angle is less than 10° or 20°) while in the stance phase, the POD 100 (e.g., the inference layer 120 of the multi-layered controller 300) may transition the current state to standing 606. In some embodiments, the standing 606 activity is a special case of the slow walk 501 activity referred to as the fumbling around (FA) activity. If the POD 100 detects a confined or limited ambulation pattern that does not show cyclical kinematic patterns, it may be managed differently compared to a true cyclical walking gait because the functional criteria or expectations of the user may be different. Standing 606 falls under that general gait activity (e.g., non-cyclical gaits), where the POD 100 may detect loading/unloading cycles on the prosthetic limb, while not necessarily being associated with a full implementation of the swing cycle's knee joint flexion-extension motion. In some embodiments, through a gait control strategy targeting both stability and agility in non-cyclical movements, the slow walk 501 gait activity differs from the fast walk 505 activity, which may implement a mobility-oriented gait strategy.

As described above, many situations and environments encountered in daily living activities may prevent a user from performing the execution of the standing-up 605 activity in an ideal or textbook context or manner. Limited physical capabilities/balance of some users, presence of external perturbations, environmental constraints are all factors that can easily cause a failure of the standing-up 605 gait activity execution. For example, the standing-up 605 activity may fail when the gait activity terminates before completing, with the user being forced to stop or deciding to stop standing-up (e.g., the user decides to return to a sitting position). When the user interrupts the standing-up 605 gait activity, the user may stop the prosthetic knee joint extension by applying sufficient weight to it, causing the control system inference layer 120 to transition directly to the yield 504 activity, which may be used to manage the stand-to-sit transfer. Once the POD 100 is operating in yield 504 gait activity, the user may be free to return to the seated position, which may cause the control system to transition back into the sit 506 activity.

In some embodiments, the standing-up 605 gait activity execution may be terminated before completing because the user is failing, or voluntarily deciding not to, maintain proper loading on the POD 100, causing the ground contact sensor 208 and control system to detect a swing phase transition. Since the standing-up 605 activity may be purely a stance-based gait activity, the POD 100 may terminate the standing-up 605 gait activity and cause the inference layer 120 to default to the slow walk 501 activity if the POD 100 detects a swing phase. In some embodiments, the POD 100 transitions to the slow walk 501 gait activity as the default activity. However, the POD 100 may immediately evaluate the measurements based on the embedded sensor signals to determine whether the slow walk 501 activity may be adequate for the situation at hand. In some embodiments, the POD 100 may transition to a variety of different gait activities after transitioning to the slow walk 501 activity from a terminated standing-up 605 gait activity, based on the specific conditions that caused the swing phase detection.

In the situation where the user may have lost balance and fallen back into the chair he or she was standing up from, the POD 100 may default to the slow walk 501 gait activity without negatively impacting the user's experience, mobility, or safety. While in slow walk 501 activity, the POD 100 may directly evaluate the POD's 100 joint and lower-limb configuration and transition directly back into the sit 506 activity if the sensor data indicates this corresponds to the user's current state.

In some embodiments, defaulting the POD's 100 activity to the slow walk 501 activity allows the POD 100 to support a direct initiation of the cyclical walking gait in the situation where the user may have voluntarily removed the load from the POD 100 while executing the sit-to-stand transition 510 to accelerate the transition execution or to mimic the way the sit-to-stand transition 510 is executed on a non-powered prosthetic knee joint. Defaulting the POD's activity to the slow walk 501 activity may allow the transition to occur smoothly without requiring the user to wait for the POD 100 to be ready or explicitly recognize the user's intention. In some embodiments, the POD 100 may transition from the slow walk 501 activity directly into the fast walk 505 activity as soon as certain conditions are met, making slow walk 501 a transitional state.

The embodiments described herein to manage the device and user interaction during the sit-to-stand transfer 510 can be applied to manage stance phase extension during stairs ascent or ramp ascent gait activities. Thus, the POD 100 may provide a flexible way to transition between the POD 100 providing net positive power or simply following the motion imparted by the user's residual limb depending on the nature of the gait activities and/or user preferences.

In some embodiments, the impedance controller 400 features are leveraged to improve swing phase execution when the user is operating in level walking gait activity (e.g., slow walk 501 activity and fast walk 505 activity). More specifically, with reference to the activities control map 500, swing phase actuator behavior associated with slow walk 501 and fast walk 505 activities can be modified from its default behavior to improve functional performance for the user.

To achieve efficient mobility, prevent stumbles, and improve efficiency in a variety of terrains, the POD 100 may restore or modify the swing phase kinematics of the POD 100. While the swing phase kinematics rely strongly on the user's residual limb and hip power when using a passive knee prosthetic joint, it is possible to use the powered POD's 100 capabilities to better support the kinematics restoration (e.g., better mimic the motion of a natural limb), while reducing the amount of effort and level of control from the user. The provision of net positive mechanical power can again provide benefits for the user, allowing the POD 100 to reduce or minimize the impact of the variability of terrains, reducing physical effort and increasing mobility, more specifically for lower-limb amputees showing limited hip power.

As described above, for the net positive power provision in the sit-to-stand gait activity, the POD 100 may use a flexible strategy for providing powered assistance in the level walking swing phases to account for the large variability in users' residual limb power, residual limb control level, and gait style preferences. Failure to provide a flexible way to manage the swing phase may result in poor acceptability and usability, which may negate the mobility and energetics benefits of using a powered knee prosthetic. The embodiments herein described provide a structured way to manage or scale the net positive power contribution of the POD 100, while maintaining the benefits, flexibility, and transparency of the force following actuator behavior.

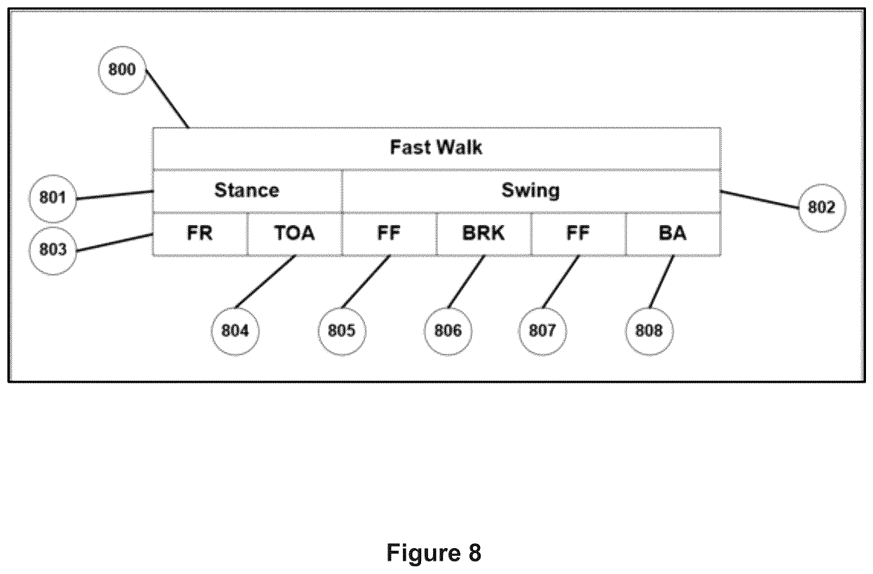

FIG. 8 presents a general schematic of the fast walk 800 gait activity management strategy. In some embodiments, the POD 100 may use the fast walk 800 gait activity to scale the amount of net positive power delivered by the POD 100 in swing phase. A similar gait activity management strategy may also be advantageously used to better support the user in other gait activities such as slow walk 501 and yield 504. The benefits of the flexibility and transparency achieved with the augmented force following strategy are significant when used in gait activities where high interaction between the POD 100 and the user occurs. On the other hand, in other gait activities, such as stairs ascent 503 or stairs down 502, which may not use or include a refined flexible and transparent behavior, satisfactory performance may be achieved using more traditional approaches such as velocity control or fixed target position control.

The fast walk 800 is a gait activity that may be associated with cyclical ambulation in continuous level walking. As with other ambulation activities observed in daily living, the fast walk 800 gait cycle may be characterized by a stance 801 phase and a swing 802 phase. In some embodiments, the stance 801 phase is further divided in two subphases, Force Rejection (FR) 803 and Toe-off Assist (TOA) 804. The typical gait cycle description may start when the prosthetic limb foot (not shown) of the POD 100 contacts the ground and weight transfer over the prosthetic limb is initiated by the user. In some embodiments, the FR 803 subphase describes the knee joint behavior from the moment where the prosthetic foot contacts the ground to the point where the user's center-of-mass has moved over the supporting limb and the hip joint starts flexing. During that subphase, the POD's 100 actuator may be or may remain locked, having the impedance controller 400 configured in Force Rejection mode, which stabilizes the knee joint, letting the compliant transmission element 210 manage the stance flexion-extension cycle.

As the user's center-of-mass progresses over the prosthetic foot, the TOA 804 subphase may be entered. In some embodiments, the TOA 804 subphase aims at proactively flexing the POD's 100 knee joint, while the POD 100 is still carrying load, to prevent toe-stubbing or forcing the user to adopt specific gait pathologies, such as hip hiking. Proactive flexion of the knee joint may also contribute to higher efficiency forward progression by increasing gait symmetry and contributing to the overall forward momentum. The TOA 804 knee flexion can be supported with a variety of motion control formulations, such as position control or velocity control, which moves the knee into flexion until the swing 802 phase is detected. When proper synergy is observed between the user and the POD 100, the TOA 804 subphase may be a strong contributor to swing flexion dynamics. However, an overly powerful TOA 804 subphase can also destabilize the user. In some embodiments, the POD 100 improves the trade-offs between balance and mobility of a user by using active or passive TOA. In some embodiments, the POD 100 can use active and/or passive TOA and is particularly suited to support swing dynamics for users that are observed to rely on a large stability support and/or have a hard time managing the stance phase proactive knee flexion. However, users that do not rely on large stability support may also benefit from the TOA.

In some embodiments, the POD 100 enters the swing 802 phase when the ground contact sensor 208 indicates that the load applied on the POD 100 has fallen below a predetermined threshold. The POD 100 may transition to or remain in the force following 805 subphase when it enters the swing 802 phase, which places the knee actuator in a low mechanical impedance state. Transitioning to the FF 805 subphases may allow the POD 100 to carry over the momentum gained during the TOA subphase 804 and pursue flexion motion. Once the POD 100 measures sufficient flexion angle, it may enter the Brake 806 (BRK) subphase, which may stop the flexion motion prior to initiating the extension motion. In some embodiments, the BRK 806 subphase allows the POD 100 to control the swing 802 phase's maximum knee flexion angle, to achieve proper toe clearance as the hip flexion cycle occurs and maintaining proper swing timing, such that the prosthetic foot may reach a position allowing the next foot strike event to take place without the user braking stride.

Once the extension velocity of the POD 100 reaches a threshold or minimum level, the BRK 806 subphase is exited and the POD's 100 subphase is changed to FF 807. Similar to the knee flexion FF 805 subphase, the knee extension FF subphase 807 may place the knee actuator in a low mechanical impedance state, which allows the POD 100 to carry over the momentum gained during the extension motion initiation, while leaving the knee in a flexible and transparent control state, where the user can influence the motion pattern directly through his or her residual limb motion. As the POD's 100 extension takes place, the Bumper Avoidance 808 (BA) subphase may be entered. In some embodiments, the BA 808 subphase is aimed at managing the knee flexion angle in late swing to prepare for the upcoming foot strike, as well as to prevent the occurrence of a terminal impact, if the knee was to reach the extension stop. Occurrence of terminal impact may reduce user comfort and can yield in loss of balance, as the impact may propagate through the POD's 100 assembly and the user's residual limb.

As described above, achieving the desired objectives of the FF 805 subphase during knee flexion of fast walk 800 may entail that the TOA 804 subphase is executed properly and allows the POD 100 to generate sufficient knee flexion momentum to power the full flexion. In the case where the TOA 804 subphase does not execute properly due to the environment, terrain, or user's clinical condition, the knee flexion motion may stop before reaching sufficient flexion angle to trigger the BRK 806 subphase, which would prevent the proper cycle to be completed. Similarly, the swing extension FF 807 subphase, may not provide sufficient speed or may expect too much direct input from the user residual limb to maintain proper extension velocity for the BA 808 subphase. In some embodiments, the POD may prevent the BRK 806 and BA 808 from failing in the situations described above by increasing the amount of net mechanical power provided by the knee actuator, while maintaining the FF actuator behavior, which may reduce the POD's 100 dependence on the user's direct contribution or the correct execution of all the subphases in the typical sequence.

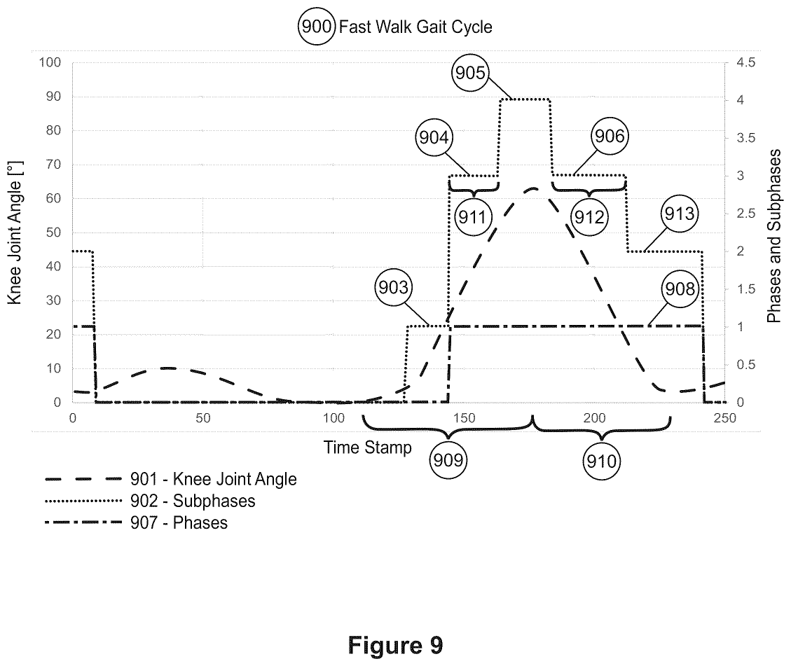

FIG. 9 further describes the fast walk gait cycle 900 and the phase/subphase mapping that may allow the POD 100 to achieve the desired outcome and make use of one or more of the embodiments disclosed herein. A single fast walk walking gait cycle measured during the system operation on a trans-femoral amputee is herein presented. Knee joint angle 901, phases 907 and subphases 902 are plotted for a single cycle, starting at foot strike. As the foot strike is detected by the POD's ground contact sensor, the phase signal 907 goes down to 0, indicating a positive stance detection. Simultaneously, the subphase 902 defaults for FR behavior, indicated by the 0 value. The knee joint angle 901 is observed to increase, before reducing again before the subphase signal value is observed to change to 1, indicative that the system has transitioned to TOA 903. After the transition to TOA 903, the knee joint angle 901 may start moving in flexion before the TOA 903 detection starts increasing flexion velocity. Phase signal 907 then transitions to value 1, indicative of Swing 908 phase detection, which in turn may cause the subphase value to go from 1 to 3, indicating that the POD's 100 is now operating in FF 904.

In the fast walk gait cycle 900, complete knee flexion motion takes places in the time range indicated by the 909 brackets, while the part executed under FF subphase is indicated by the 911 markers and may include approximately one-third of the complete motion range. As described above, once the knee reaches sufficient flexion angle, the POD 100 may transition to the BRK 905 subphase, where the knee is decelerated to meet a threshold (maximum) flexion angle, before being reaccelerated in extension. Once the knee reaches sufficient extension velocity, the FF 906 subphase may be entered allowing the knee to complete a large part of the extension motion cycle 910 in transparent mode, under FF 906 actuator behavior as indicated by the 912 markers. As the knee nears full extension under significant velocity BA 913 subphase may be entered taking over control of the extension motion and eventually leaving the knee in a lightly flexed configuration, in preparation for the coming foot strike. It should be understood that a user's fast walk gait cycle may vary from the gait cycle 900 based on the user's preferences and characteristics, as well as the current terrain and task.

FIG. 10 presents the same fast walk gait cycle 1000 as introduced in FIG. 9, but FIG. 10 adds the knee torque signal 1001 to the signals already introduced above: knee joint angle 1002, phase signal 1004 and subphase signal 1003. Again, the swing flexion motion takes places as per indicated by the 1007 bracket, while swing extension is shown by 1008 brackets. The swing flexion FF 1005 subphase maps to the matching area on the knee measured torque signal 1001, as well as the swing extension FF 1006 subphase. From the knee measured torque curve presented on FIG. 10, it can be observed that 1 in the swing flexion FF 1005 subphase, impact of the force following low mechanical impedance control makes the measured torque drop to a level close to 0 Nm quickly after the subphase is entered. According to the strategy and the controller configuration used for force following behavior creation, the actuator may move the actuator output in such a way to maintain the measured knee torque close to null, reducing or minimizing the perceived impedance. Similar behavior is observed during the swing extension FF 1006, with the knee measured torque rapidly reducing once the POD 100 transitions to the force following control.

In some embodiments, the force following behavior is modified to manage the net positive power delivery performed by the POD 100 when operating in the force following subphase of the fast walk activity. In some embodiments, to reduce the direct contribution expectation from the user while maintaining the typical force following transparency characteristic, the knee torque measurement provided by the compliant transmission sensor may be voluntarily biased at the impedance controller 400 level, artificially modifying the feedback signal 406. By adding a fixed quantity to the actual measured torque, the force following behavior may move under small perturbations more easily. Through proper adjustment of the offset based on user preference, or type of user or clinical characteristic, the amount of net positive power provided by the POD 100 can be adjusted so that the POD's 100 operation is improved or even optimized for efficiency and to provide the user a high level of efficiency, safety, and flexibility. It is to be noted that this methodology generates inherently different results from applying a larger gain to the torque feedback signal. As a general example of the difference in both approaches, applying a larger gain to a small torque value would not yield the desired behavior as the dynamic response would also be modified.

In contrast to other control schemes that may result in a similar reduction of the user perceived mechanical impedance, the disclosed embodiments do not rely on a priori defined control inputs. Some of the embodiments disclosed herein may be used to inject net positive power, while the user remains in control of the POD's 100 behavior since the input for the control system may be the mechanical interaction measured by the torque sensor. To improve the POD's 100 reliability and remove possible drawbacks associated with the creation of a positive feedback system, the POD 100 may bound the offset that is applied to the measured torque signals when operating in force following mode.

In some embodiments, the POD 100 may determine the range of allowed torque sensor signal offset for a given embodiment of a POD 100 by matching the threshold (maximum) offset value to the measured system friction torque or actuation dead band. Thus, the POD 100 may be reactive to occurrences generated by the user's direct command or perturbation, without creating enough torque feedback for the POD 100 to move faster than the user's command. In the case where the POD's 100 actuator presents different non-linearities in flexion or extension, separate rules may be used to manage the sensor signal offset separately. However, in the case where the direction of motion is already known from the task or operational conditions, the system may not manage the direction in which the offset is to be applied.

In some embodiments, the POD 100 makes use of separate torque sensor signal offsets for the swing flexion FF 1005 and swing extension FF 1006. Due to the effect of the gravity on the knee joint suspended load combined with the fact that swing phase extension can be more easily affected by the user through motion of his or her residual limb, most lower- limb amputees may benefit from increased support in swing flexion, while the support can be lowered for swing extension.

Alternatively, or in addition, the POD 100 may not apply the offset to the measured torque signal for the entire duration of the FF subphase of the fast walking gait activity, but may apply the offset (only) when the torque output set-point is found to be below a predetermined threshold or minimum value. Again, this method allows the POD 100 to reduce or minimize the dead band and provides more uniform perceived impedance to the user when operating at a low torque set-point, which is typical from swing phase operation. It is to be noted that while this modification of the force following control scheme is well suited for the fast walk gait activity and has been herein described in view of this last gait activity, its use can also be generalized to other gait activities without departing from the intent of the disclosed embodiments.

In some cases, another mechanism can be used to manage the stand-to-sit transition. While the use of the yield 504 gait activity coupled to entering conditions based on duration and amplitude of the measured knee torque are shown to provide natural and highly functional performance, the user may wait for a sufficient time period (e.g., 0.25 seconds, 0.5 seconds, 1 second, 2 seconds, longer than 2 seconds, or any amount of time in-between) to elapse for the gait activity to be entered. Additionally, the use of the interaction torque as the gait activity transition input signal may be a limitation for users with less control or strength over the residual limb hip.

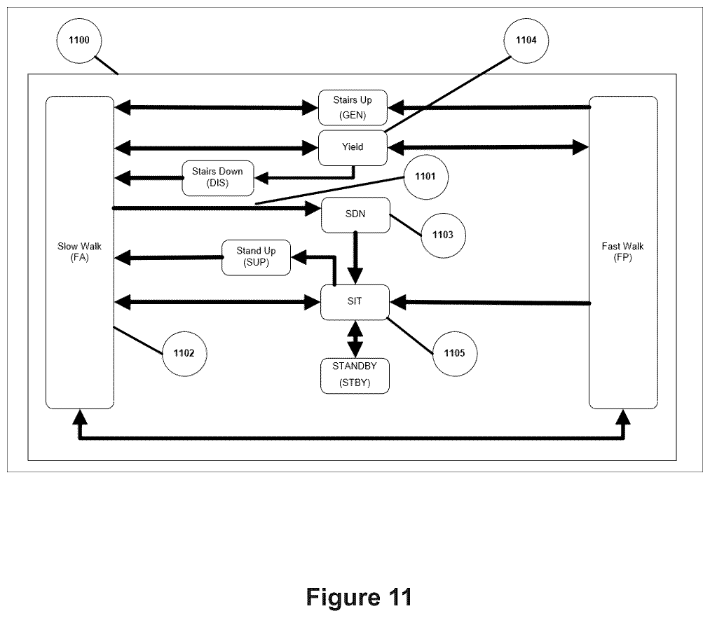

FIG. 11 presents a high-level block diagram of a control map 1100 illustrating the locomotion activities and transitions of one embodiment of a control system for a POD 100 where the SDN (sit-down) 1103 gait activity is managed separately from the yield 1104 gait activity. The control map (e.g., the gait activities and transitions map) 1100 presented in FIG. 11 may overcome the limitations for users with low residual limb hip strength and/or may be better suited for users who prefer more dynamic transitions. In some embodiments, each gait activity and gait activity transition of the control map 1100 may correspond to one or more actuator behaviors. It is to be understood that this control map 1100 is provided to illustrate the nature and operation of one embodiment of the POD 100 and is non-limiting.

As shown in FIG. 11, in some embodiments, the POD 100 may perform the stand-to-sit activity through the SDN 1103 gait activity. It should be understood that FIG. 11 is a non-limiting example, and that some embodiments of the POD 100 may perform the stand-to-sit activity through the SDN 1103 gait activity while having different numbers of gait activities, configurations, and/or relationships between gait activities used to implement the specific gait control system.

In contrast to the control map 500, management of the SDN 1103 gait activity in control map 1100 may be separated from the management of the other dissipative tasks and/or gait activities performed by the POD 100. While the yield 504 gait activity was previously used to manage the SDN 1103 gait activity, it may no longer be the case in this embodiment of the control map 1100. The yield 1104 gait activity may differ from the yield 504 gait activity (e.g., yield 1104 may cover a narrower spectrum of gait activities than yield 504 since SDN 1103 may cover some gait activities that are covered by yield 504). In some embodiments, the other gait activities presented in the control map 1100 may be similar to the gait activities described in previous embodiments (e.g., FIG. 5).

Under the gait control system control map 1100, the SDN 1103 is a general gait activity to support the user's transition from standing to sitting. Similar to what may be implemented in yield 1104 or 504 gait activities previously described, the POD's 100 functional task is directed towards controlled lowering of the user's weight as the POD 100 flexes under load until the seated position is reached and the load is removed from the POD 100. In some embodiments, the SDN 1103 activity may provide sufficient torque for the knee to flex to the seated position in a steady and smooth motion and prevent the knee from collapsing under the user during the sitting motion. In some embodiments, the POD may use torque control (e.g., maintaining the knee torque within a target knee torque range), proportional control, position control, velocity control (e.g., maintaining the rotational velocity below a threshold rotational velocity), or any other control scheme described herein to manage the flex of the knee during the SDN 1103 activity. In certain cases, the POD 100 may use the impedance controller 400 to control the flexion of the knee joint during the SDN activity similar to a force rejection behavior.

In some embodiments, the SDN 1103 gait activity exits to sit 1105 gait activity when the swing phase is detected following the removal of the load on the POD 100 or when the flexion angle or flexion velocity stabilizes to a value inside of predetermined boundaries (e.g., an upper and lower threshold). The latter case allows the POD 100 to manage the inherent variability in the knee flexion angle when the user is seated, which is both a function of the seat characteristics coupled to the user's lower-limb segment length and segment length ratio.

In some embodiments, the SDN 1103 gait activity is entered from the slow walk 1102 gait activity (e.g., transition 1101), which may be a representative use scenario for the users having a low general activity level or presenting residual limb hip control or strength limitations. Some embodiments provide a method to manage the gait activity transition from slow walk 1102 to SDN 1103 when the user initiates the SDN 1103 gait activity, such that there is no perceived delay in entering the SDN 1103 gait activity and obtaining the desired knee joint function (e.g., provide knee torque to slow down flexion during sitting motion). The SDN 1103 gait activity may also reduce the amount of residual hip strength and/or control expected of the user.

In some embodiments, the POD 100 may detect the entry conditions for the SDN 1103 gait activity without using the knee joint measured interaction torque by leveraging sensor data indicative of the lower limb configuration and general user posture and motion as described herein. This approach may allow the POD 100 to avoid the overlap in sensing data when using the measured knee torque, where similar knee torque amplitudes are descriptive of both the SDN 1103 gait activity and level walking gait activity. In some embodiments, the POD 100 uses the loading time profile to distinguish between the sensing data overlap. In some cases, using the loading time profile creates a burden on the user and decreases the overall device usability for this specific user scenario.

Due to the specific nature of the stand-to-sit functional task, the POD 100 may detect the entry conditions of the SDN 1103 gait activity (e.g., the stand-to-sit transition) by using one or more measurements. In some cases, the entry conditions for the SDN 1103 activity may include (1) the absolute angle of the second limb is less than a threshold (e.g., less than zero degrees), (2) the rotational velocity of the second limb member is within a threshold velocity range, and/or (3) the load distribution satisfies a load distribution threshold (e.g., the user's weight is towards the heel instead of the toe and/or more weight is over the heel than the toe). Any combination of the entry conditions described herein may be used to accommodate a user's needs and preferences.

In some embodiments, the POD 100 may detect the entry conditions of the SDN 1103 gait activity by evaluating the user's posture (e.g., the absolute angle and/or position of the thigh, or other body part, such as the user's chest, etc.) combined with the user's motion (e.g., thigh velocity, thigh rotational velocity, thigh acceleration, and rotational acceleration of user's chest, hip, residual limb, and/or second limb, as well as, the knee rotational velocity and acceleration). In some embodiments, the POD 100 may initiate the SDN 1103 activity when the user moves his or her center of mass posteriorly at the beginning of the sitting motion (e.g., in preparation for the knee flexion that may lower the user's center of mass). The POD 100 may use (1) the positioning of the user's center-of-mass behind the user's feet and/or (2) the backward movement of the user's center-of-mass to establish a unique set of entry conditions for the SDN 1103 activity (e.g., entry conditions that distinguish the sitting activity from other similar movements).

Using the embedded IMU capabilities and/or other sensors, the POD 100 may determine the absolute angle (e.g., including the position and rotation) of the second limb member (e.g., the shank) in the sagittal plane. The POD 100 may use the absolute orientation and/or motion of the second limb member to determine the location of the user's center of mass with respect to the base of support (e.g., the user's foot) with high accuracy. For example, since the knee does not allow for significant extension motion, the user's center of mass may be considered posterior as soon as the shank's absolute angle in the sagittal plane indicates a posterior tilt (e.g., a negative angle). In some embodiments, the POD 100 may determine that the user's center of mass is behind his or her base of support if the absolute angle of the second limb is less than a threshold angle. The threshold angle may be −10, −5, 0°, 5°, 10°, etc. Similarly, the POD 100 may determine that the user's center of mass is moving backward if there is no forward motion by the user (e.g., the user is stationary or not walking forward).

To reinforce the user's posture evaluation certainty and prevent overlap of various activities where the second limb (e.g., shank) tilt angle may also be found to show posterior tilt, other conditions may be used to verify in which direction the user's posture may be changing. For example, the rate of change of the second limb's sagittal plane tilt angle (e.g., rotational velocity) may provide a strong indication of how the user's posture is evolving in real time or over a certain time period. To prevent overlap of conditions that include similar rotation of the second limb, such as when the user is walking backward, a threshold (maximum) limit or range may be used to distinguish the conditions or actions by measuring the amplitude of the shank's rotational velocity in the sagittal plane. For example, the SDN 1103 gait activity is characterized by a lower rate of change of the shank's sagittal tilt angle than what can be observed in walking backward, allowing the POD 100 to easily distinguish between the two gait activities. For example, if the POD 100 determines that the magnitude of the rotational velocity for the second limb is less than a first or upper threshold, the POD can enter the SDN 1103 activity. In some embodiments, the upper threshold for the magnitude of the rotational velocity may be ˜300°/s, ˜200°/s, ˜100°/s, ˜50°/s, ˜25°/s, etc. The POD 100 may also, have a second (lower) threshold for the magnitude of the rotational velocity as an entry condition for the SDN 1103 activity. The POD may use the minimum threshold to avoid entering the SDN 1103 activity due to noise or insignificant user motions. For example, if the POD 100 determines that the magnitude of the rotational velocity for the second limb is greater than the second or lower threshold, the POD can enter the SDN 1103 activity. In some embodiments, the second or lower threshold for the magnitude of the rotational velocity may be at ˜5°/s, ˜10°/s, ˜20°/s, ˜50°/s, ˜75°/s, etc. In some cases, the POD 100 may use a magnitude range for the rotational velocity of the second limb as an entry condition for the SDN 1103 activity (e.g., rotation velocity within the range will cause the POD 100 to enter the SDN 1103 activity). For example, the magnitude range for the rotational velocity may have a lower threshold of ˜10°/s and an upper rotational velocity threshold of ˜200°/s.

In some embodiments, a third sensor stream can be used to eliminate further overlap with other commonly encountered use scenarios and to confirm that the user intends to sit-down. For example, in a use scenario where the user is rocking back and forth on his or her feet while standing, it can be considered possible that the conditions listed above could be met, while the user intent is not to sit down. To further eliminate these false positive detection scenarios, it is possible to test the ground contact sensor signal to get a relative measure of how far behind the vertical projection of the user's center-of-mass is compared to the user's feet (e.g., to determine whether the user's weight is distributed more towards the user's heels than the user's toes). More specifically, in the case where a ground contact sensor of the type disclosed in U.S. Nonprovisional patent application Ser. No. 17/638,493 allows the POD 100 to estimate the sagittal plane location of the vertical projection of the user's center-of-mass using multiple sensors located in the antero-posterior direction, the POD 100 may use this sensor data information to strengthen the intent detection.

In some embodiments, it is possible to compare the difference in measured vertical ground reaction force on the posterior sensor(s) to the one registered to the anterior sensor(s) to a predetermined threshold. When the difference between the posterior and anterior vertical loading is observed to exceed the predetermined threshold (e.g., the load on the posterior load sensor(s) is larger than the load on the anterior load sensor(s) by 25%), the POD 100 may determine that the user's center-of mass is sufficiently posterior to the location of the prosthetic foot to make it unavoidable for the SDN 1103 gait activity to occur. For example, as the center-of-mass location moves more posteriorly, the user will eventually lose balance and fall backward unless the SDN 1103 gait activity takes place.

It is to be noted that the sensitivity of the SDN 1103 gait activity can be adjusted by varying the thresholds used on the three signals introduced above. The thresholds and sensitivity for any of these signals may be adjusted by the POD's 100 processes or by the user. For example, one or more of the thresholds may be modified based on the terrain, environment, and/or user's physical state and characteristics to improve the responsiveness, efficiency, and safety of the POD 100. While the shank segment sagittal plane tilt angle and tilt angle rate of change are bounded by other gait activities with similar characteristics, it is possible to use the difference of loading between the anterior and posterior vertical ground reaction forces threshold or minimum value to adjust the gait activity detection sensitivity in a more free manner. In some embodiments, any of these signals may also have duration thresholds.

In some embodiments, the POD 100 may primarily use one of the signals, while using the other two signals as secondary confirmations (e.g., once the first signal has satisfied its corresponding threshold). Alternatively, the POD 100 could predominantly utilize any two sensor signals, while employing the third signal as a supplementary confirmation. In some embodiments, the POD 100 uses the three signals (e.g., orientation of the second limb, rotational velocity of the second limb, and load distribution) equally. As described with other entry conditions, the entry conditions may occur asynchronously. For example, one or more of the entry conditions may initiate a detection window. During the detection window, the POD 100 may initiate the SDN 1103 gait activity (e.g., the stand-to-sit transition) if one or more remaining conditions are satisfied.

In some embodiments, a threshold or minimum difference of 60 units (e.g., a 60 analog-to-digital unit difference between two analog-to-digital converters) between the posterior and the anterior measured ground reaction force vertical components is considered adequate to indicate the transition to SDN 1103 gait activity. This threshold value was also observed to work properly when associated with the shank segment's sagittal plane tilt angle being more posterior than vertical (e.g., meaning that the top part of the shank segment is posterior to the prosthetic foot) and the rate of change of the shank segment's sagittal plane tilt angle to be inferior to 10°/sec. In some embodiments, the difference threshold value is a percentage. For example, the threshold value may be a 1% difference, 10% difference, 30% difference, 40% difference, 50% difference, more than 50%, or any percentage in-between, between the posterior and the anterior measured ground reaction forces.

### Additional Embodiments

In embodiments of the present disclosure, a prosthetic or orthotic device may be in accordance with any of the following clauses:
Clause 1. A prosthetic or orthotic device comprising: a first limb member; a second limb member coupled to the first limb member at a joint; an actuator coupled to the first limb member and the second limb member and configured to actuate the first limb member relative to the second limb member; and a controller configured to control the actuator, the controller further configured to: based at least in part on a determination that an angle between the first limb member and the second limb member satisfies an angle parameter threshold, select a standing-up mode for a controller mode of the controller, wherein in the standing-up mode, the controller causes the actuator to exhibit an extension behavior during a first portion of a stance phase and exhibit a braking behavior during a second portion of the stance phase.
Clause 2. The prosthetic or orthotic device of Clause 1, wherein the prosthetic or orthotic device further comprises a ground contact sensor and a torque sensor.
Clause 3. The prosthetic or orthotic device of Clause 2, wherein the controller is further configured to: based at least in part on a determination that the prosthetic or orthotic device is under a load that satisfies a load threshold, select the standing-up mode for the controller mode of the controller.
Clause 4. The prosthetic or orthotic device of any preceding clause, wherein the prosthetic or orthotic device further comprises one or more position sensors.
Clause 5. The prosthetic or orthotic device of any preceding clause, wherein the angle parameter threshold is a first parameter threshold, and wherein the second portion of the stance phase begins when the controller determines that the angle between the first limb member and the second limb member satisfies a second angle parameter threshold.
Clause 6. The prosthetic or orthotic device of any preceding clause, wherein the controller is further configured to: based at least in part on a determination that a torque value does not satisfies a torque threshold, cause the extension behavior to correspond to a force following behavior, and based at least in part on a determination that the torque value satisfies the torque threshold, cause the extension behavior to correspond to a power injection behavior.
Clause 7. The prosthetic or orthotic device of any preceding clause, wherein the controller is further configured to: based at least in part on a determination that an absolute orientation of the first limb member satisfies an orientation threshold, select the standing-up mode for the controller mode of the controller.
Clause 8. The prosthetic or orthotic device of any preceding clause, wherein the controller is further configured to: based at least in part on a determination that a torque value satisfies a torque threshold, cause the extension behavior to correspond to a power injection behavior.
Clause 9. A prosthetic or orthotic device comprising: a first limb member; a second limb member coupled to the first limb member at a joint; an actuator coupled to the first limb member and the second limb member and configured to actuate the first limb member relative to the second limb member; and a controller configured to control the actuator via a control map, wherein the control map comprises gait activities and gait activity transitions, wherein each of the gait activities and the gait activity transitions correspond to one or more actuator behaviors, and wherein the controller is further configured to: based at least in part on a determination that one or more entry conditions of a gait activity are satisfied, select a gait activity transition to transition from a first gait activity to a second gait activity, wherein the second gait activity is determined based on the one or more entry conditions that were satisfied.
Clause 10. The prosthetic or orthotic device of any preceding clause, wherein the prosthetic or orthotic device further comprises a ground contact sensor and a torque sensor.
Clause 11. The prosthetic or orthotic device of Clause 10, wherein one of the gait activity transitions is a sit-to-stand transition, wherein the controller uses the sit-to-stand transition to transition from a sit gait activity to a stand gait activity.
Clause 12. The prosthetic or orthotic device of Clause 11, wherein a first entry condition of the one or more entry conditions corresponds to a torque value satisfying a torque threshold, wherein the controller is further configured to: based at least in part on a determination that the first entry condition was satisfied, select the sit-to-stand transition.
Clause 13. The prosthetic or orthotic device of Clause 11, wherein the sit-to-stand transition, has at least two subphases, wherein each of the subphases corresponds to different actuator behaviors.
Clause 14. The prosthetic or orthotic device of any preceding clause, wherein the prosthetic or orthotic device further comprises one or more position sensors.
Clause 15. The prosthetic or orthotic device of Clause 14, wherein a first entry condition of the one or more entry conditions corresponds to an angle between the first limb member and the second limb member satisfying an angle parameter threshold, wherein the controller is further configured to: based at least in part on a determination that the first entry condition was satisfied, select a sit-to-stand transition.
Clause 16. The prosthetic or orthotic device of Clause 11, wherein the gait activities comprise at least a slow walk gait activity and a fast walk gait activity.
Clause 17. The prosthetic or orthotic device of Clause 16, wherein the fast walk gait activity corresponds to a first set of actuator behaviors during a stance phase, and a second set of actuator behaviors during a swing phase.
Clause 18. The prosthetic or orthotic device of Clause 16, wherein the controller may use the slow walk gait activity as an intermediate gait activity between two other gait activities.
Clause 19. The prosthetic or orthotic device of Clause 9, wherein at least one of the gait activities has at least two subphases, wherein each of the at least two subphases correspond to different actuator behaviors.
Clause 20. The prosthetic or orthotic device of Clause 9, wherein the gait activities comprise at least a yield gait activity and wherein the controller uses the yield gait activity as an intermediate gait activity between two gait activities.
Clause 21. The prosthetic or orthotic device of Clause 20, wherein the controller uses the yield gait activity as an intermediate gait activity between two gait activities.
Clause 22. A prosthetic or orthotic device comprising: a first limb member; a second limb member coupled to the first limb member at a joint; an actuator coupled to the first limb member and the second limb member and configured to actuate the first limb member relative to the second limb member; a torque sensor; and a controller configured to control the actuator via a control map, wherein the control map comprises gait activities and gait activity transitions, wherein each of the gait activities and the gait activity transitions correspond to one or more actuator behaviors, wherein the gait activities comprise at least a slow walk gait activity and a fast walk gait activity, and wherein the fast walk gait activity corresponds to a first set of one or more actuator behaviors during a stance phase, and a second set of one or more actuator behaviors during a swing phase, and wherein the controller is further configured to: based at least in part on a determination that one or more entry conditions of a gait activity are satisfied, select a gait activity transition to transition from a first gait activity to a second gait activity, wherein the second gait activity is determined based on the one or more entry conditions that were satisfied.
Clause 23. The prosthetic or orthotic device of any preceding clause, wherein the first set of one or more actuator behaviors corresponding to the fast walk gait activity comprise at least a force rejection behavior and a toe-off assist behavior.
Clause 24. The prosthetic or orthotic device of any preceding clause, wherein the second set of one or more actuator behaviors corresponding to the fast walk gait activity comprise at least a force following behavior and a breaking behavior.
Clause 25. The prosthetic or orthotic device of Clause 24, wherein the second set of one or more actuator behaviors corresponding to the fast walk gait activity further comprise at least a bumper avoidance behavior.
Clause 26. The prosthetic or orthotic device of any preceding clause, wherein the prosthetic or orthotic device further comprises a ground contact sensor.
Clause 27. The prosthetic or orthotic device of any preceding clause, wherein one of the gait activity transitions is a sit-to-stand transition, wherein the controller uses the sit-to-stand transition to transition from a sit gait activity to a stand gait activity.
Clause 28. The prosthetic or orthotic device of Clause 27, wherein a first entry condition of the one or more entry conditions corresponds to a torque value satisfying a torque threshold, wherein the controller is further configured to: based at least in part on a determination that the first entry condition was satisfied, select the sit-to-stand transition.
Clause 29. The prosthetic or orthotic device of Clause 28, wherein the sit-to-stand transition, has at least two subphases, wherein each of the subphases corresponds to different actuator behaviors.
Clause 30. The prosthetic or orthotic device of any preceding clause, wherein the prosthetic or orthotic device further comprises one or more position sensors.
Clause 31. The prosthetic or orthotic device of Clause 30, wherein a first entry condition of the one or more entry conditions corresponds to an angle between the first limb member and the second limb member satisfying an angle parameter threshold, wherein the controller is further configured to: based at least in part on a determination that the first entry condition was satisfied, select a sit-to-stand transition.
Clause 32. The prosthetic or orthotic device of any preceding clause, wherein the controller may use the slow walk gait activity as an intermediate gait activity between two other gait activities.
Clause 33. The prosthetic or orthotic device of any preceding clause, wherein at least one of the gait activities has at least two subphases, wherein each of the subphases corresponds to different actuator behaviors.
Clause 34. The prosthetic or orthotic device of Clause 22, wherein the gait activities comprise at least a yield gait activity.
Clause 35. The prosthetic or orthotic device of Clause 34, wherein the controller uses the yield gait activity as an intermediate gait activity between two gait activities.
Clause 36. A prosthetic or orthotic device, comprising: a first limb member; a second limb member coupled to the first limb member at a joint; an actuator coupled to the first limb member and the second limb member and configured to actuate the first limb member relative to the second limb member; and a controller configured to: receive a measurement of an absolute angle of the second limb member, and select a sit-down mode for a controller mode of the controller based at least in part on a determination that the absolute angle of the second limb member satisfies an angle parameter threshold, wherein in the sit-down mode, the controller causes the actuator to support a user's transition from a standing position to a sitting position by providing resistance to a rotation of the second limb member relative to the first limb member.
Clause 37. The prosthetic or orthotic device of Clause 36, wherein the controller is configured to determine that the absolute angle of the second limb member satisfies and angle parameter threshold based at least in part on a determination that the absolute angle of the second limb member is less than 0°.
Clause 38. The prosthetic or orthotic device of Clause 36 or Clause 37, wherein in the sit-down mode, the controller uses torque control, and wherein the controller is further configured to: control the actuator to maintain a measured knee torque within a target knee torque range.
Clause 39. The prosthetic or orthotic device of any one of Clauses 36 to 38, wherein the controller is further configured to: determine a rotational velocity of the second limb member, wherein the controller is configured to select the sit-down mode based at least in part on a determination that the rotational velocity of the second limb member is within a velocity rotational range.
Clause 40. The prosthetic or orthotic device of any one of Clauses 36 to 39, wherein the prosthetic or orthotic device further comprises one or more posterior load sensors, one or more anterior load sensors, and a torque sensor.
Clause 41. The prosthetic or orthotic device of Clause 40, wherein the controller is further configured to: select the sit-down mode for the controller mode of the controller based at least in part on a determination that a first load on the one or more posterior load sensors is larger than a second load on the one or more anterior load sensors.
Clause 42. A prosthetic or orthotic device comprising: a first limb member; a second limb member coupled to the first limb member at a joint; an actuator coupled to the first limb member and the second limb member and configured to actuate the first limb member relative to the second limb member; and a controller configured to control the actuator via a control map, wherein the control map comprises gait activities and gait activity transitions, wherein each of the gait activities and the gait activity transitions correspond to one or more actuator behaviors, wherein one of the gait activity transitions is a stand-to-sit transition, wherein the controller uses the stand-to-sit transition to transition from a sit gait activity to a stand gait activity, and wherein the controller is further configured to: transition from a first gait activity to a second gait activity based at least in part on a determination that one or more entry conditions corresponding to the second gait activity are satisfied.
Clause 43. The prosthetic or orthotic device of Clause 42, wherein the prosthetic or orthotic device further comprises an IMU.
Clause 44. The prosthetic or orthotic device of Clause 42 or Clause 43, wherein the prosthetic or orthotic device further comprises one or more torque sensors.
Clause 45. The prosthetic or orthotic device of Clause 44, wherein a first set of one or more actuator behaviors corresponding to the stand-to-sit transition comprises using torque control, wherein the controller is further configured to: control an actuator output to maintain a measured knee torque within a target knee torque range.
Clause 46. The prosthetic or orthotic device of any one of Clauses 42 to 45, wherein a first set of one or more actuator behaviors corresponding to the stand-to-sit transition comprises using velocity control, wherein the controller is further configured to: control an actuator output to maintain a measured rotational velocity of the first limb member relative to the second limb member (or joint) below a threshold rotational velocity.
Clause 47. The prosthetic or orthotic device of Clause 43, wherein a first entry condition of the one or more entry conditions corresponds to a rotation value of the second limb member satisfying a rotation threshold, wherein the controller is further configured to: based at least in part on a determination that the first entry condition was satisfied, select the stand-to-sit transition.
Clause 48. The prosthetic or orthotic device of Clause 47, wherein a second entry condition of the one or more entry conditions corresponds to a rotational velocity value of the second limb member satisfying a rotational velocity range, wherein the controller is further configured to: based at least in part on a determination that the second entry condition was satisfied, select the stand-to-sit transition.
Clause 49. The prosthetic or orthotic device of any one of Clauses 42 to 48, wherein the prosthetic or orthotic device further comprises one or more posterior load sensors and one or more anterior load sensors.
Clause 50. The prosthetic or orthotic device of Clause 48 or Clause 49, wherein a third entry condition of the one or more entry conditions corresponds to a load distribution profile where a first load on the one or more posterior load sensors is larger than a second load on the one or more anterior load sensors, wherein the controller is further configured to: based at least in part on a determination that the third entry condition was satisfied, select the stand-to-sit transition.

Clause 51. A prosthetic or orthotic device comprising: a first limb member; a second limb member coupled to the first limb member at a joint; an actuator coupled to the first limb member and the second limb member and configured to actuate the first limb member relative to the second limb member; an encoder; and a controller configured to control the actuator according to a control map, wherein the control map comprises gait activities and gait activity transitions, wherein the gait activities and the gait activity transitions correspond to one or more actuator behaviors, wherein the gait activities comprise at least a standing-up activity, and wherein the controller is further configured to: use a rotational velocity control scheme to control the one or more actuator behaviors during at least a first portion of the standing-up activity based at least in part on a determination that a rotational extension velocity of the joint is less than a first rotational extension velocity threshold.

Clause 52. The prosthetic or orthotic device of Clause 51, wherein the controller is further configured to: cause the one or more actuator behaviors of the standing-up activity to correspond to a force following behavior during at least a second portion of the standing-up activity based at least in part on a determination that the rotational extension velocity of the joint is more than a second rotational extension velocity threshold.

Clause 53. The prosthetic or orthotic device of Clause 51 or Clause 52, wherein the rotational velocity control scheme is a velocity closed-loop control scheme with a corresponding target rotational extension velocity, wherein the controller is further configured to: cause the joint to extend at the target rotational extension velocity when the rotational velocity control scheme is in use.

Clause 54. The prosthetic or orthotic device of Clause 53, wherein the rotational extension velocity threshold is equal to the target rotational extension velocity.

Clause 55. The prosthetic or orthotic device of any one of Clauses 51 to 54, wherein the controller is further configured to: cause the one or more actuator behaviors of the standing-up activity to correspond to a force following behavior during at least a second portion of the standing-up activity based at least in part on a determination that an angle of the joint is less than an angle threshold.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “May,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A prosthetic or orthotic device, comprising:
a first limb member;
a second limb member coupled to the first limb member at a joint;
an actuator coupled to the first limb member and the second limb member and configured to actuate the first limb member relative to the second limb member; and
a controller configured to:
receive a measurement of an absolute angle of the second limb member, and
select a sit-down mode for a controller mode of the controller based at least in part on a determination that the absolute angle of the second limb member satisfies an angle parameter threshold, wherein in the sit-down mode, the controller causes the actuator to support a user's transition from a standing position to a sitting position by providing resistance to a rotation of the second limb member relative to the first limb member.

2. The prosthetic or orthotic device of claim 1, wherein the controller is configured to determine that the absolute angle of the second limb member satisfies and angle parameter threshold based at least in part on determination that the absolute angle of the second limb member is less than 0°.

3. The prosthetic or orthotic device of claim 1, wherein in the sit-down mode, the controller uses torque control, and wherein the controller is further configured to:
control the actuator to maintain a measured knee torque within a target knee torque range.

4. The prosthetic or orthotic device of claim 1, wherein the controller is further configured to:
determine a rotational velocity of the second limb member,
wherein the controller is configured to select the sit-down mode based at least in part on a determination that the rotational velocity of the second limb member is within a rotational velocity range.

5. The prosthetic or orthotic device of claim 1, wherein the prosthetic or orthotic device further comprises one or more posterior load sensors, one or more anterior load sensors, and a torque sensor.

6. The prosthetic or orthotic device of claim 5, wherein the controller is further configured to:
select the sit-down mode for the controller mode of the controller based at least in part on a determination that a first load on the one or more posterior load sensors is larger than a second load on the one or more anterior load sensors.

7. A prosthetic or orthotic device comprising:
a first limb member;
a second limb member coupled to the first limb member at a joint;
an actuator coupled to the first limb member and the second limb member and configured to actuate the first limb member relative to the second limb member; and
a controller configured to control the actuator via a control map, wherein the control map comprises gait activities and gait activity transitions, wherein each of the gait activities and the gait activity transitions correspond to one or more actuator behaviors, wherein one of the gait activity transitions is a stand-to-sit transition, wherein the controller uses the stand-to-sit transition to transition from a sit gait activity to a stand gait activity, and wherein the controller is further configured to:
transition from a first gait activity to a second gait activity based at least in part on a determination that one or more entry conditions corresponding to the second gait activity are satisfied.

8. The prosthetic or orthotic device of claim 7, wherein the prosthetic or orthotic device further comprises an IMU.

9. The prosthetic or orthotic device of claim 7, wherein the prosthetic or orthotic device further comprises one or more torque sensors.

10. The prosthetic or orthotic device of claim 9, wherein a first set of one or more actuator behaviors corresponding to the stand-to-sit transition comprises using torque control, wherein the controller is further configured to:
control an actuator output to maintain a measured knee torque within a target knee torque range.

11. The prosthetic or orthotic device of claim 7, wherein a first set of one or more actuator behaviors corresponding to the stand-to-sit transition comprises using velocity control, wherein the controller is further configured to:
control an actuator output to maintain a measured rotational velocity of the first limb member relative to the second limb member below a threshold rotational velocity.

12. The prosthetic or orthotic device of claim 8, wherein a first entry condition of the one or more entry conditions corresponds to a rotation value of the second limb member satisfying a rotation threshold, wherein the controller is further configured to:
based at least in part on a determination that the first entry condition was satisfied, select the stand-to-sit transition.

13. The prosthetic or orthotic device of claim 12, wherein a second entry condition of the one or more entry conditions corresponds to a rotational velocity value of the second limb member satisfying a rotational velocity range, wherein the controller is further configured to:
based at least in part on a determination that the second entry condition was satisfied, select the stand-to-sit transition.

14. The prosthetic or orthotic device of claim 13, wherein the prosthetic or orthotic device further comprises one or more posterior load sensors and one or more anterior load sensors.

15. The prosthetic or orthotic device of claim 14, wherein a third entry condition of the one or more entry conditions corresponds to a load distribution profile where a first load on the one or more posterior load sensors is larger than a second load on the one or more anterior load sensors, wherein the controller is further configured to:
based at least in part on a determination that the third entry condition was satisfied, select the stand-to-sit transition.

16. A prosthetic or orthotic device comprising:
a first limb member;
a second limb member coupled to the first limb member at a joint;
an actuator coupled to the first limb member and the second limb member and configured to actuate the first limb member relative to the second limb member;
an encoder; and
a controller configured to control the actuator according to a control map, wherein the control map comprises gait activities and gait activity transitions, wherein the gait activities and the gait activity transitions correspond to one or more actuator behaviors, wherein the gait activities comprise at least a standing-up activity, and wherein the controller is further configured to:
use a rotational velocity control scheme to control the one or more actuator behaviors during at least a first portion of the standing-up activity based at least in part on a determination that a rotational extension velocity of the joint is less than a first rotational extension velocity threshold.

17. The prosthetic or orthotic device of claim 16, wherein the controller is further configured to:
cause the one or more actuator behaviors of the standing-up activity to correspond to a force following behavior during at least a second portion of the standing-up activity based at least in part on a determination that the rotational extension velocity of the joint is more than a second rotational extension velocity threshold.

18. The prosthetic or orthotic device of claim 16, wherein the rotational velocity control scheme is a velocity closed-loop control scheme with a corresponding target rotational extension velocity, wherein the controller is further configured to:
cause the joint to extend at the target rotational extension velocity when the rotational velocity control scheme is in use.

19. The prosthetic or orthotic device of claim 18, wherein the rotational extension velocity threshold is equal to the target rotational extension velocity.

20. The prosthetic or orthotic device of claim 16, wherein the controller is further configured to:
cause the one or more actuator behaviors of the standing-up activity to correspond to a force following behavior during at least a second portion of the standing-up activity based at least in part on a determination that an angle of the joint is less than an angle threshold.
